(19) United States
(12) Patent Application Publication
(10) Pub. No.: US 2026/0235586 A1
(43) Pub. Date: Aug. 13, 2026

(54) COMPOSITIONS AND METHODS RELATED TO APTAMER-BASED SENSORS

(71) Applicant: North Carolina State University, Raleigh, NC (US)

(72) Inventors: Yi Xiao, Raleigh, NC (US); Juan Canoura, Raleigh, NC (US)

(21) Appl. No.: 19/156,509

(22) PCT Filed: Feb. 14, 2024
(86) PCT No.: PCT/US2024/015706

§ 371 Date: Aug. 14, 2025

Related U.S. Application Data
(60) Provisional application No. 63/484,901, filed on Feb. 14, 2023.

(51) Int. Cl. G01N 33/53; C12N 15/10; C12N 15/115; G01N 33/94

(52) U.S. Cl. CPC G01N 33/5308; C12N 15/1048; C12N 15/115; G01N 33/9486; C12N 2310/16

(57) ABSTRACT

The present disclosure provides compositions and methods related to aptamer-based sensors. In particular, the present disclosure provides aptamer-based sensors, and related detection assays, that are capable of binding fentanyl (and derivatives and analogs thereof) at nanomolar concentrations in biological samples in a manner that is rapid and specific.

### CROSS REFERENCE TO RELATED APPLICATIONS

This application claims priority to and the benefit of U.S. Provisional Patent Application No. 63/484,901 filed Feb. 14, 2023, which is incorporated herein by reference in its entirety and for all purposes.

### GOVERNMENT SUPPORT

The invention was made with government support under grant number 2019-DU-BX-0024 awarded by the National Institute of Justice. The government has certain rights in the invention.

### SEQUENCE LISTING STATEMENT

The contents of the electronic sequence listing filed herewith, titled “NCSU_41482_601_SequenceListing”, having a file size of 36,301 bytes, and created Feb. 14, 2024, is herein incorporated by reference in its entirety.

### FIELD

The present disclosure provides compositions and methods related to aptamer-based sensors. In particular, the present disclosure provides aptamer-based sensors, and related detection assays, that are capable of binding fentanyl (and derivatives and analogs thereof) in a manner that is rapid, specific, and sensitive.

### BACKGROUND

Opioids are one of the most frequently prescribed medications in the United States, primarily for the treatment of moderate to severe pain, and are commonly applied in surgical settings. One of the most commonly employed opioids is the synthetic opioid fentanyl, which is used in critical care for sedation and pain management during surgery, intubation, or in other contexts where relief for severe pain is needed. Fentanyl is also prescribed as an analgesic for discharged patients. The popularity of fentanyl can be attributed to its simple synthesis, low cost, and high potency, which allows for low dosing. Fentanyl activates the μ-opioid receptor to provide pain relief with 100-fold greater potency than morphine. Dosing fentanyl is challenging, as its high potency leads to a narrow therapeutic window, requiring careful drug monitoring to maintain therapeutic effects while minimizing dangerous side effects-most critically, respiratory depression. Analgesia typically employs serum concentrations of fentanyl ranging from 0.6-9 nM, while 30-90 nM serum fentanyl is associated with anesthetic effects. Reaching and maintaining therapeutic levels of fentanyl are typically accomplished using general dosing recommendations for relatively healthy patients, but dosing is problematic for infants (<1 year), elderly patients (>50 years), and especially in those with life-threatening conditions such as morbid obesity, kidney failure, sepsis, or cardiovascular disease, and improper dosing in this context can create high risk of mortality. Moreover, in recent years, fentanyl has been increasingly abused for recreational purposes and is a primary contributor to the opioid crisis in the United States. It is frequently used as an adulterant in illicit substances such as heroin, cocaine, and methamphetamine, and is even used as a primary drug of abuse. As a result, fentanyl is the fourth most abused drug and the number one cause for drug-related overdose deaths in the US as of 2022.

Given the narrow therapeutic window of fentanyl, as well as its complex and rapid pharmacokinetics and toxicokinetics, there is a critical need for assays/tools that enable rapid and frequent measurement of this drug in biological samples. Current approaches based on liquid chromatography-tandem mass spectrometry are time-consuming, require sample pretreatment and trained personnel, and are limited to a specialized laboratory setting. Immunoassays are a promising replacement given their great sensitivity, and several enzyme-linked immunosorbent assays (ELISAs) are currently available for use in oral fluid, blood, and urine. These assays detect fentanyl with limits of detection (LODs) ranging from 0.9-3 nM, but they are time-consuming, requiring at least 30-60 min to obtain results. Moreover, these assays have been shown to cross-react with non-opioid interferents, including 3,4-methyl enedioxymethamphetamine (MDMA), methamphetamine, and diphenhydramine. As an alternative, lateral-flow immunoassays allow for rapid on-site detection of fentanyl within 5 min, albeit with reduced sensitivity (30-600 nM). However, these assays are solely intended for use with urine samples. Therefore, there remains a need for fentanyl assays that are rapid, sensitive, and specific and which can detect fentanyl in several different biological matrices—serum, oral fluid, and urine—ideally at on-site or point-of-care settings.

### SUMMARY

Embodiments of the present disclosure include a single-stranded nucleic acid molecule capable of binding fentanyl, or a derivative or analog thereof, comprising a nucleic acid sequence that is at least 50% identical to the following: X₁GCTTGGX₂TAGGTCGGGX₃TTGGGTX₄CGGX₅X₆; wherein X₁ is A, T, C, or G; X₂ is A, T, C, or G; X₃ is A, T, C, or G; X₄ is A, T, C, or G; X₅ is A, T, C, or G; and X₆ is A, T, C, or G (SEQ ID NO: 1).

In some embodiments, X₁ is T or A; X₂ is G or T; X₃ is T or C, X₄ is T or G, X₅ is C or A; and X₆ is G or T (SEQ ID NO: 2). In some embodiments, the nucleic acid molecule comprises a nucleic acid sequence that is at least 50% identical to any of SEQ ID NOs: 5, 8, 10, 13, 17, 19, 25, 27, 28, and 29. In some embodiments, the nucleic acid molecule comprises a K_{D} that is less than about 500 nM.

Embodiments of the present disclosure also include a single-stranded nucleic acid molecule capable of binding fentanyl, or a derivative or analog thereof, comprising a nucleic acid sequence that is at least 50% identical to the following: X₁X₂AATGGX₃AGCATTGGTX₄X₅X₆X₇X₈X₉X₁₀X₁₁X₁₂X₁₃X₁₄X₁₅X₁₆; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ A, T, C, or G; X₁₁ is A, T, C, or G; X₁₂ is A, T, C, or G; X₁₃ is A, T, C, or G; X₁₄ is A, T, C, or G; X₁₅ is A, T, C, or G; and X₁₁ is A, T, C, or G (SEQ ID NO: 3).

In some embodiments, X₁ is A or G; X₂ is A or G; X₃ is C or A, X₄ is G or T, X₅ is A or G; X₆ is C or G; X₇ is T or G; X₈ is G or T; X₉ is G or A; X₁₀ G or T; X₁₁ is T or G; X₁₂ is T or C; X₁₃ is G or T; X₁₄ is C or T; X₁₅ is T or C; and X₁₁ is T or C (SEQ ID NO: 4). In some embodiments, the nucleic acid molecule comprises a nucleic acid sequence that is at least 50% identical to any of SEQ ID NOs: 6, 9, 11, 15, and 26. In some embodiments, the nucleic acid molecule comprises a K_{D} that is less than about 500 nM.

In accordance with the above embodiments, the nucleic acid molecule is capable of binding fentanyl, or a derivative or analog thereof, under physiological conditions.

In some embodiments, the nucleic acid molecule comprises a detection moiety.

In some embodiments, the nucleic acid molecule is in solution or attached to a substrate.

In some embodiments, the fentanyl derivative or analog is selected from the group consisting of: acetyl fentanyl, furanyl fentanyl, acrylfentanyl, butyryl fentanyl, valeryl fentanyl, cyclopropyl fentanyl, methoxyacetyl fentanyl, p-fluoro fentanyl, o-methyl furanyl fentanyl, o-methoxy furanyl fentanyl, p-methoxy furanyl fentanyl, p-methoxy butyryl fentanyl, FIBF, alpha-methyl thiofentanyl, 3,4-dimethoxy fentanyl, beta-hydroxy thiofentanyl, cis-3-methyl fentanyl, trans-3-methyl fentanyl, cis-3-methyl thiofentanyl, trans-3-methyl thiofentanyl, sufentanil, alfentanil, remifentanil, benzylfentanyl, N-benzyl furanyl norfentanyl, N-benzyl m-fluoro norfentanyl, N-benzyl o-fluoro norfentanyl, N-benzyl phenyl norfentanyl, norfentanyl, and 4-ANPP.

Embodiments of the present disclosure also include a vector comprising any of the nucleic acid sequences of the aptamers described herein.

Embodiments of the present disclosure also include a method of detecting fentanyl, or a derivative or analog thereof. In accordance with these embodiments, the method includes combining any of the nucleic acid molecules of the aptamers described herein comprising a fluorescent moiety with a quencher-labeled nucleic acid molecule that is at least partially complementary to the nucleic acid molecules of the aptamers described herein to form a quenched composition; and exposing the quenched composition to a sample comprising or suspected of comprising fentanyl, or a derivative or analog thereof. In some embodiments, the presence of the fentanyl, or a derivative or analog thereof, in the sample displaces the quencher-labeled nucleic acid molecule, thereby producing a fluorescent signal proportional to the concentration of the fentanyl, or a derivative or analog thereof, in the sample.

Embodiments of the present disclosure also include a method of detecting fentanyl, or a derivative or analog thereof. In accordance with these embodiments, the method includes combining any of the nucleic acid molecules of the aptamers described herein with a reporter compound that binds to the nucleic acid molecules of the aptamers described herein to form a complexed composition; and exposing the complexed composition to a sample comprising or suspected of comprising fentanyl, or a derivative or analog thereof. In some embodiments, the presence of the fentanyl, or a derivative or analog thereof, in the sample displaces the reporter compound, thereby allowing the reporter compound to form detectable aggregates proportional to the concentration of the fentanyl, or a derivative or analog thereof, in the sample.

Embodiments of the present disclosure also include a method of detecting fentanyl, or a derivative or analog thereof. In accordance with these embodiments, the methods include immobilizing any of the nucleic acid molecules of the aptamers described herein to an electrically conductive substrate, wherein the nucleic acid molecules of the aptamers described herein comprise a redox tag, to form a detection sensor; and exposing the detection sensor to a sample comprising or suspected of comprising fentanyl, or a derivative or analog thereof. In some embodiments, the presence of the fentanyl, or a derivative or analog thereof, in the sample binds the nucleic acid molecules of the aptamers described herein thereby producing an electrochemical signal proportional to the concentration of the fentanyl, or a derivative or analog thereof, in the sample.

In some embodiments, the sample is a biological sample from a human subject. In some embodiments, the biological sample is a saliva sample, a urine sample, a blood sample, a serum sample, a plasma sample, a fecal sample, a CSF sample, or a tissue sample.

In some embodiments of these methods, the fentanyl derivative or analog is selected from the group consisting of: acetyl fentanyl, furanyl fentanyl, acrylfentanyl, butyryl fentanyl, valeryl fentanyl, cyclopropyl fentanyl, methoxyacetyl fentanyl, p-fluoro fentanyl, o-methyl furanyl fentanyl, o-methoxy furanyl fentanyl, p-methoxy furanyl fentanyl, p-methoxy butyryl fentanyl, FIBF, alpha-methyl thiofentanyl, 3,4-dimethoxy fentanyl, beta-hydroxy thiofentanyl, cis-3-methyl fentanyl, trans-3-methyl fentanyl, cis-3-methyl thiofentanyl, trans-3-methyl thiofentanyl, sufentanil, alfentanil, remifentanil, benzylfentanyl, N-benzyl furanyl norfentanyl, N-benzyl m-fluoro norfentanyl, N-benzyl o-fluoro norfentanyl, N-benzyl phenyl norfentanyl, norfentanyl, and 4-ANPP.

Embodiments of the present disclosure also include a method of identifying a nucleic acid aptamer capable of binding fentanyl, or a derivative or analog thereof. In accordance with these embodiments, the method includes (i) exposing a library of candidate aptamers to fentanyl, or a derivative or analog thereof; (ii) collecting candidate aptamers exhibiting rapid target association kinetics; and (iii) repeating steps (i) and (ii) for at least one addition round of selection to generate a plurality of high-affinity candidate aptamers.

In some embodiments, the method further comprises sequencing the plurality of high-affinity candidate aptamers.

In some embodiments, the method further comprises characterizing the plurality of high-affinity candidate aptamers using isothermal titration calorimetry (ITC).

In some embodiments, the method further comprises assessing binding specificity of the plurality of high-affinity candidate aptamers using an exonuclease-based fluorescence assay.

### BRIEF DESCRIPTION OF THE DRAWINGS

FIGS. 1A-1E: Selection of DNA aptamer that binds to the synthetic opioid fentanyl under physiological conditions. (A) Scheme of the library-immobilized SELEX process used here to isolate aptamers. (B) The percentage of pool eluted each round by fentanyl per concentration of fentanyl. (C) The pool affinity of the round 10 pool determined using a gel elution assay. (D) A pathway of the SELEX process, with pool affinity of each target fraction post PCR amplification and the target fractions used as PCR amplicons to create a new pool for the next round of selection highlighted in blue. (E) The pool binding affinity of the three different target fractions of round 13. For all pool affinity measurements, since the binding curves suggest the presence of two distinct populations of aptamers with divergent affinities, all binding curves were fit with a modified two-site Langmuir equation with two KDs reported.

FIGS. 2A-2B: Binding affinity characterization of fentanyl aptamers isolated via SELEX. (A) Plots used to classify four different types of aptamers depending on their growth pattern during SELEX: exponential (Category I, E1), linear (Category II, L1), peaked (Category III, P1), and declining (Category IV, D2). (B) Sequence and ITC-derived fentanyl-binding affinity of the most abundant aptamers from category 1, 2, and 3 (SEQ ID NOs: 5-29).

FIGS. 3A-3D: A strand displacement fluorescence assay for detecting fentanyl in human serum. (A) Aptamer L3 labeled with a terminal Cy5 tag (L3-Cy5) hybridizes to a quencher-labeled (Iowa Black, IB) 14-nt complementary DNA strand (cDNA-IB), thereby quenching fluorescence. Fentanyl binding to the aptamer displaces cDNA-IB, resulting in recovery of fluorescence. (B) Optimization of cDNA-IB concentration to attain 80% quenching of Cy5 fluorescence. (C) Calibration curves for fentanyl detection in buffer (black), 25% (blue) or 50% (red) human serum. (D) Linear range of the assay.

FIGS. 4A-4E: Detection of fentanyl using an aptamer-based dye displacement assay. (A) The small organic dye MTC is initially complexed with the P2 aptamer in a monomeric or dimeric state. In the presence of fentanyl, the dye is displaced into aqueous solution, where it forms J-aggregates with a distinct absorption profile. (B) Optimization of the concentration of aptamer (P2) for a fixed concentration of 1.5 μM MTC. The plot depicts absorbance of MTC monomers (λₘₐₓ=590 nm) and J-aggregates (λₘₐₓ=650 nm) as a function of aptamer concentration. (C) Absorbance spectra of MTC after mixing aptamer-dye complexes (8 μM P2, 1.5 μM MTC) with various concentrations of fentanyl in buffer. (D) Corresponding calibration curves and (E) linear ranges for the assay in buffer (black) and 50% saliva (red).

FIGS. 5A-5B: Assessing the affinity and specificity of various fentanyl aptamers using an exonuclease digestion assay to identify optimal aptamers for E-AB sensor fabrication. Resistance values of (A) 25 different aptamers in the presence of 25 μM or 250 μM fentanyl and (B) aptamers E2, P2, and L1 for various interferents and their predicted secondary structures. The red-shaded area represents a resistance value of ±0.05, or about 10% cross-reactivity relative to fentanyl, which represents no ligand binding.

FIGS. 6A-6E: Detection of fentanyl using E-AB sensors fabricated with E2-34-MB. (A) E-AB sensors consist of an aptamer tagged with methylene-blue covalently bound to gold electrode surface. Target binding to the aptamer causes the aptamer to undergo a conformational change, which alters the distance of the methylene blue tag from the electrode surface, and hence electric current when scanned via voltammetry. (B) Square wave voltammograms produced with the E-AB sensors in the presence of various concentrations of fentanyl. (C) Calibration curves of the E-AB sensors for fentanyl detection in buffer (blue), 50% human urine (red), 50% human saliva (black), and 50% human serum (gold), with signals at low target concentrations shown in D. (E) Cross-reactivity of the E-AB sensor to various interferents relative to 10 μM fentanyl, with the red-shaded area representing 10% cross-reactivity.

FIGS. 7A-7B: Representative structures and consensus sequences of nucleic acid aptamers capable of binding fentanyl (and derivatives and analogs thereof) from (A) E2 family and (B) E1 family.

FIG. 8: Chemical structures of fentanyl and fentanyl analogs used in the various embodiments of the present disclosure.

FIGS. 9A-9C: Representative graphical data of the cross-reactivity of three fentanyl aptamers with respect to the various fentanyl analogs indicated in FIG. 8. FIG. 9A includes cross-reactivity data for E2 aptamer; FIG. 9B includes cross-reactivity data for aptamer L1; FIG. 9C includes cross-reactivity data for aptamer P2.

FIG. 10: Chemical structures of interferents employed in counter-SELEX.

FIGS. 11A-11B: Specificity of the round 10 combined pool determined by gel elution assay. (A) Polyacrylamide gel electrophoresis (PAGE) results depict the ligand elution profile, with lanes representing samples of the pool eluted with various ligands. (B) Determination of the cross-reactivity of the round 10 fentanyl pool against 25 μM fentanyl; 250 μM procaine, (+)-methamphetamine, lidocaine, diphenhydramine, benzocaine, cocaine, ethylone, heroin, naloxone, naltrexone, oxycodone, methylphenidate, methadone amphetamine, cis-tramadol, quinine, or chlorpromazine; or 100 μM clomipramine, citalopram, alprazolam, amoxapine, lorazepam, clonazepam. Cross-reactivity is calculated relative to 25 μM fentanyl.

FIGS. 12A-12C: Binding affinity of the round 10 (A) fraction 1, (B) fraction 2, and (C) fraction 3 pools. All fractions were PCR amplified and then analyzed via gel-elution assay. Since the binding curves suggest the presence of two distinct populations of aptamers with divergent affinities, all binding curves were fitted with a modified two-site Langmuir equation with two K_{D}s reported.

FIGS. 13A-13C: Binding affinity of the round 11 (A) fraction 1, (B) fraction 2, and (C) fraction 3 pools. All fractions were PCR amplified and then analyzed via gel-elution assay. As above, all binding curves were fitted with a modified two-site Langmuir equation with two K_{D}s reported.

FIGS. 14A-14C: Binding affinity of the round 12 (A) fraction 1, (B) fraction 2, and (C) fraction 3 pools. All fractions were PCR amplified and analyzed via gel-elution assay. As above, all binding curves were fit with a modified two-site Langmuir equation with two K_{D}s reported.

FIGS. 15A-15B: Specificity of the fraction 1 of Round 13 pool determined by gel elution assay. (A) PAGE results depict the elution profile with various ligands. (B) Cross-reactivity of the fraction 1 of Round 13 against 250 μM fentanyl; 250 μM procaine, (+)-methamphetamine, lidocaine, diphenhydramine, benzocaine, cocaine, ethylone, heroin, naloxone, naltrexone, oxycodone, methylphenidate, methadone, amphetamine, cis-tramadol, quinine, or chlorpromazine; or 100 μM clomipramine, citalopram, alprazolam, amoxapine, lorazepam, or clonazepam. Cross-reactivity is calculated relative to 250 μM fentanyl.

FIGS. 16A-16B: High-throughput sequencing analysis of fractions 1 and 2 of round 10 and fraction 1 of round 11, 12, and 13. (A) Pool diversity. (B) Abundance of sequences from the final enriched R13 pool belonging to the following categories: 1) exponential growth rate, 2) linear growth rate, 3) peaked in population, and 4) consistently decreasing in population.

FIGS. 17A-17F: Characterization of fentanyl binding affinity of six aptamer candidates from category 1 using ITC. Top panels display the heat generated from each titration of fentanyl into (A) E1, (B) E2, (C) E3, (D) E4, (E) E5, and (F) E6. Bottom panels show the integrated heat of each titration after correcting for the heat of dilution of the titrant.

FIGS. 18A-18D: Characterization of fentanyl binding affinity of four aptamer candidates from category 1 using ITC. Top panels display the heat generated from each titration of fentanyl into (A) E7, (B) E8, (C) E9, and (D) E10. Bottom panels show the integrated heat of each titration after correcting for the heat of dilution of the titrant.

FIGS. 19A-19E: Characterization of fentanyl binding affinity of five aptamer candidates from category 3 using ITC. Top panels display the heat generated from each titration of fentanyl into (A) P1, (B) P2, (C) P3, (D) P4, and (E) P5. Bottom panels show the integrated heat of each titration after correcting for the heat of dilution of the titrant.

FIGS. 20A-20F: Characterization of fentanyl binding affinity of six aptamer candidates from category 2 using ITC. Top panels display the heat generated from each titration of fentanyl into (A) L1, (B) L2, (C) L3, (D) L4, (E) L5, and (F) L6. Bottom panels show the integrated heat of each titration after correcting for the heat of dilution of the titrant.

FIGS. 21A-21D: Characterization of fentanyl binding affinity of four aptamer candidates from category 2 using ITC. Top panels display the heat generated from each titration of fentanyl into (A) L7, (B) L8, (C) L9, and (D) L10. Bottom panels show the integrated heat of each titration after correcting for the heat of dilution of the titrant.

FIG. 22: Digestion time-course of P2 by a mixture of Exo III and Exo I in the presence of 10 μM fentanyl, delta9-tetrahydrocannabinol, and AB-FUBINACA; 50 μM clomipramine, citalopram, amoxapine, alprazolam, clonazepam and lorazepam; or 250 μM for benzocaine, procaine, diphenhydramine, lidocaine, quinine, chlorpromazine, cocaine, (+)-methamphetamine, amphetamine, (+)-pseudoephedrine, ethylone, heroin, oxycodone, naloxone, naltrexone, methylphenidate, methadone, and cis-tramadol.

FIG. 23: Absorbance spectra of MTC (1.5 μM) after mixing with P2 at various concentrations (0-20 μM). Decreased J-aggregate absorbance (650 nm) and increased monomer (590 nm) and dimer (550 nm) absorbance were observed with increasing concentrations of P2.

FIG. 24: Absorbance spectra of MTC obtained by mixing complexes formed with 8 μM P2 and 1.5 μM MTC with various concentrations of fentanyl (0-50 μM) in 50% saliva.

FIGS. 25A-25B: Absorbance spectra of 1.5 μM MTC mixed with various concentrations of fentanyl (0-50 μM) in the absence of P2 aptamer in (A) selection buffer and (B) 50% saliva.

FIGS. 26A-26J: Digestion time-course of fentanyl-binding aptamer candidates (A) E1, (B) E2, (C) E3, (D) E4, (E) E5, (F) E6, (G) E7, (H) E8, (I) E9, and (J) E10 in the presence of 0 (black), 25 (maroon), or 250 μM (red) fentanyl.

FIGS. 27A-27J: Digestion time-course of fentanyl-binding aptamer candidates (A) L1, (B) L2, (C) L3, (D) L4, (E) L5, (F) L6, (G) L7, (H) L8, (I) L9, and (J) L10 in the presence of 0 (black), 25 (maroon), or 250 μM (red) fentanyl.

FIGS. 28A-28E: Digestion time-course of fentanyl-binding aptamer candidates (A) P1, (B) P2, (C) P3, (D) P4, and (E) P5 in the presence of 0 (black), 25 (maroon), or 250 μM (red) fentanyl.

FIGS. 29A-29C: Screening the specificity of fentanyl-binding aptamers from category 1 against interferents using an exonuclease digestion assay. Plots show resistance value obtained from time-course digestion of (A) E1, (B) E4, and (C) E9 in the presence of 10 μM fentanyl, delta9-tetrahydrocannabinol, and AB-FUBINACA; 50 μM clomipramine, citalopram, amoxapine, alprazolam, clonazepam, and lorazepam; or 250 μM other interferents.

FIGS. 30A-30C: Screening the specificity of fentanyl-binding aptamers from category 2 and category 3 against interferents using an exonuclease digestion assay. Plots show resistance value obtained from time-course digestion of (A) L5, (B) P1, and (C) P3 in the presence of 10 μM fentanyl, delta9-tetrahydrocannabinol, and AB-FUBINACA; 50 μM clomipramine, citalopram, amoxapine, alprazolam, clonazepam, and lorazepam; or 250 μM other interferents.

FIG. 31: Screening the specificity of fentanyl-binding aptamers from category 3 against interferents using an exonuclease digestion assay. Plots show resistance value obtained from time-course digestion of P5, in the presence of 10 μM fentanyl, delta9-tetrahydrocannabinol, and AB-FUBINACA; 50 μM clomipramine, citalopram, amoxapine, alprazolam, clonazepam, and lorazepam; or 250 μM other interferents.

FIG. 32: Identification of major digestion products based on PAGE analysis of a time-course digestion of E2 with 250 μM fentanyl. NuPACK-predicted secondary structure of parent aptamer E2 and hypothetical structure of the major digestion product E2-40 are provided at left and right, respectively.

FIG. 33: Characterization of fentanyl-binding affinity of E2-34 using ITC. Top panels display the heat generated from each titration of fentanyl into E2-34. Bottom panels show the integrated heat of each titration after correcting for the heat of dilution of the titrant.

FIG. 34: Circular dichroism spectra of E2-34 in the absence (black) and presence (red) of 10 μM fentanyl to confirm target-binding induced conformational change.

FIGS. 35A-35B: Optimization of (A) surface coverage of electrochemical aptamer-based (E-AB) sensors constructed with E2-34-MB for detection of fentanyl, and (B) performance of the E-AB sensors modified with an optimal surface coverage of 4.0 pmol/cm² E2-34-MB at various frequencies for detection of 25 μM fentanyl.

FIGS. 36A-36C: Square-wave voltammetry (SWV) data for E2-34-MB modified E-AB sensors in the presence of 0-50 μM fentanyl in (A) 50% saliva, (B) 50% urine, and (C) 50% serum.

FIGS. 37A-37I: SWV spectra collected in buffer with (red) or without (black) 100 μM procaine, benzocaine, diphenhydramine, lidocaine, quinine, cocaine, (+)-methamphetamine, amphetamine, or ethylone using E2-34 fabricated E-AB sensors.

FIGS. 38A-38I: SWV spectra collected in buffer with (red) or without (black) 100 μM of heroin, oxycodone, naloxone, naltrexone, methylphenidate, methadone, or cis-tramadol, or 50 μM clomipramine or citalopram using E2-34 fabricated E-AB sensors.

FIGS. 39A-39D: SWV spectra collected in buffer with (red) or without (black) 50 μM amoxapine, alprazolam, clonazepam, or lorazepam using E2-34 modified E-AB sensors.

### DETAILED DESCRIPTION

Aptamers are oligonucleotide-based bio-affinity reagents isolated in vitro via systematic evolution of ligands by exponential enrichment (SELEX). They have several advantages over antibodies, including low cost, ease of modification via chemical synthesis, tunable binding profiles, reversible denaturation, and simplicity of engineering to introduce reporting functionality. This has led to the development of several aptamer-based sensors capable of rapid, sensitive, and specific detection of small molecules in various biofluids, including whole blood, serum, urine, oral fluid, and cerebrospinal fluid. As described further herein, the experiments conducted implemented a new, modified library-immobilized SELEX approach termed “Kinetic-SELEX” to isolate high affinity fentanyl-binding aptamers under ionic conditions mimicking biological samples. Alongside thermodynamic selection pressure introduced via manipulating target concentrations, Kinetic-SELEX uses target-binding kinetics as an additional selection pressure mechanism to rapidly isolate aptamers with superior affinity. This method is inspired by the discovery that target elution time has a significant effect on the binding properties of enriched aptamer pools. Using this finding, aptamers were collected with rapid target association kinetics (kₒₙ) that typically elute in the first fraction of target elution for subsequent rounds of selection, which allowed for the selective enrichment of high affinity aptamers. After selection and sequencing, the binding affinity of aptamer candidates were characterized using isothermal titration calorimetry (ITC), and their binding specificity was screened using an exonuclease-based fluorescence assay. Three of the isolated aptamers were used to fabricate various fentanyl sensors, and they demonstrated sensitive and specific target detection in different biosamples. The highest affinity aptamer (K_{D}=11 nM) was used to construct a fluorescent strand-displacement sensor capable of detecting 2.5 nM fentanyl in 25% serum. Another, highly-specific aptamer was used to develop a colorimetric dye-displacement assay that could achieve a LOD of 100 nM fentanyl in 50% oral fluid. Finally, an electrochemical aptamer-based (E-AB) sensor was constructed using another highly specific aptamer, which was capable of detecting fentanyl within seconds with a LOD of 10 nM in 50% serum, urine, and oral fluid. These sensors have utility in compliance monitoring of fentanyl in urine, detection of recent fentanyl use in oral fluid, and detection of fentanyl in serum for guiding the dosing of challenging patients and assessing drug overdose.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

The term “aptamer” generally refers to a nucleic acid molecule (e.g., oligonucleotide) of a single defined sequence or a mixture of said nucleic acid molecules, wherein the mixture retains the properties of binding specifically to a target molecule. Thus, as used herein “aptamer” denotes both singular and plural sequences of oligonucleotides. The term “aptamer” generally refers to a single-stranded oligonucleotide that is capable of binding to a protein or other molecule, and thereby modulating function. In some embodiments, the aptamers of the present disclosure are DNA molecules, including any derivatives thereof (e.g., modified nucleotide bases). In some embodiments, the aptamers of the present disclosure are RNA molecules, including any derivatives thereof (e.g., modified nucleotide bases). In some embodiments, the aptamers of the present disclosure are combinations of DNA and RNA molecules, including any derivatives thereof, and can include modified nucleotide bases.

The term “single-stranded” oligonucleotides generally refers to those oligonucleotides that contain a single covalently linked series of nucleotide residues.

The terms “oligomers” or “oligonucleotides” include RNA or DNA molecules (e.g., nucleic acid molecules) of more than one nucleotide in either single chain or duplex form and specifically includes short sequences such as dimers and trimers, in either single chain or duplex form, which can be intermediates in the production of the specifically binding oligonucleotides. “Modified” forms used in candidate pools contain at least one non-native residue. “Oligonucleotide” or “oligomer” is generic to polydeoxyribonucleotides (containing 2′-deoxy-D-ribose or modified forms thereof), such as DNA, to polyribonucleotides (containing D-ribose or modified forms thereof), such as RNA, and to any other type of polynucleotide which is an N-glycoside or C-glycoside of a purine or pyrimidine base, or modified purine or pyrimidine base or abasic nucleotides. “Oligonucleotide” or “oligomer” can also be used to describe artificially synthesized polymers that are similar to RNA and DNA, including, but not limited to, oligos of peptide nucleic acids (PNA).

The terms “binding activity” and “binding affinity” generally refer to the tendency of a ligand molecule to bind or not to bind to a target. The energetics of these interactions are significant in “binding activity” and “binding affinity” because they can include definitions of the concentrations of interacting partners, the rates at which these partners are capable of associating, and the relative concentrations of bound and free molecules in a solution.

“Complementary” refers to the characteristic of two or more structural elements (e.g., peptide, polypeptide, nucleic acid, small molecule, etc.) of being able to hybridize, dimerize, or otherwise form a complex with each other. For example, a “complementary peptide and polypeptide” are capable of coming together to form a complex. Complementary elements may require assistance to form a complex (e.g., from interaction elements), for example, to place the elements in the proper conformation for complementarity, to co-localize complementary elements, to lower interaction energy for complementation, etc.

As used herein, the terms “nucleotide sequence identity” or “nucleic acid sequence identity” refers to the presence of identical nucleotides at corresponding positions of two polynucleotides. Polynucleotides have “identical” sequences if the sequence of nucleotides in the two polynucleotides is the same when aligned for maximum correspondence (e.g., in a comparison window). Sequence comparison between two or more polynucleotides is generally performed by comparing portions of the two sequences over a comparison window to identify and compare local regions of sequence similarity. The comparison window is generally from about 20 to 200 contiguous nucleotides. The “percentage of sequence identity” for polynucleotides, such as about 5, 10, 15, 20, 25, 30, 35, 40, 45, 50, 55, 60, 65, 70, 75, 80, 85, 90, 95, 98, 99 or 100 percent sequence identity, can be determined by comparing two optimally aligned sequences over a comparison window, wherein the portion of the polynucleotide sequence in the comparison window can include additions or deletions (i.e., gaps) as compared to the reference sequence for optimal alignment of the two sequences. In some embodiments, the percentage is calculated by: (a) determining the number of positions at which the identical nucleic acid base occurs in both sequences; (b) dividing the number of matched positions by the total number of positions in the window of comparison; and (c) multiplying the result by 100. Optimal alignment of sequences for comparison can also be conducted by computerized implementations of known algorithms, or by visual inspection. Readily available sequence comparison and multiple sequence alignment algorithms are, respectively, the Basic Local Alignment Search Tool (BLAST) and ClustalW/ClustalW2/Clustal Omega programs available on the Internet (e.g., the website of the EMBL-EBI). Other suitable programs include, but are not limited to, GAP, BestFit, Plot Similarity, and FASTA, which are part of the Accelrys GCG Package available from Accelrys, Inc. of San Diego, Calif., United States of America. See also Smith & Waterman, 1981; Needleman & Wunsch, 1970; Pearson & Lipman, 1988; Ausubel et al., 1988; and Sambrook & Russell, 2001.

### 2. Aptamers

In accordance with the various embodiments of the present disclosure, described herein are methods and compositions pertaining to aptamer-based sensors. In particular, the present disclosure provides aptamer-based sensors, and related detection assays, that are capable of binding fentanyl (and derivatives and analogs thereof) in a manner that is rapid, specific, and sensitive.

In some embodiments of the present disclosure, the compositions described herein include a single-stranded nucleic acid molecule capable of binding fentanyl, or a derivative or analog thereof, comprising a nucleic acid sequence that is at least 50% identical to the following: X₁GCTTGGX₂TAGGTCGGGX₃TTGGGTX₄CGGX₅X₆; wherein X₁ is A, T, C, or G; X₂ is A, T, C, or G; X₃ is A, T, C, or G; X₄ is A, T, C, or G; X₅ is A, T, C, or G; and X₆ is A, T, C, or G (SEQ ID NO: 1). In some embodiments, the nucleic acid is at least 60% identical to the following: X₁GCTTGGX₂TAGGTCGGGX₃TTGGGTX₄CGGX₅X₆; wherein X₁ is A, T, C, or G; X₂ is A, T, C, or G; X₃ is A, T, C, or G; X₄ is A, T, C, or G; X₅ is A, T, C, or G; and X₆ is A, T, C, or G (SEQ ID NO: 1). In some embodiments, the nucleic acid is at least 70% identical to the following: X₁GCTTGGX₂TAGGTCGGGX₃TTGGGTX₄CGGX₅X₆; wherein X₁ is A, T, C, or G; X₂ is A, T, C, or G; X₃ is A, T, C, or G; X₄ is A, T, C, or G; X₅ is A, T, C, or G; and X₆ is A, T, C, or G (SEQ ID NO: 1). In some embodiments, the nucleic acid is at least 80% identical to the following: X₁GCTTGGX₂TAGGTCGGGX₃TTGGGTX₄CGGX₅X₆; wherein X₁ is A, T, C, or G; X₂ is A, T, C, or G; X₃ is A, T, C, or G; X₄ is A, T, C, or G; X₅ is A, T, C, or G; and X₆ is A, T, C, or G (SEQ ID NO: 1). In some embodiments, the nucleic acid is at least 90% identical to the following: X₁GCTTGGX₂TAGGTCGGGX₃TTGGGTX₄CGGX₅X₆; wherein X₁ is A, T, C, or G; X₂ is A, T, C, or G; X₃ is A, T, C, or G; X₄ is A, T, C, or G; X₅ is A, T, C, or G; and X₆ is A, T, C, or G (SEQ ID NO: 1).

In some embodiments, X₁ is T or A; X₂ is G or T; X₃ is T or C, X₄ is T or G, X₅ is C or A; and X₆ is G or T (SEQ ID NO: 2). In accordance with these embodiments, the nucleic acid molecule comprises a nucleic acid sequence that is at least 50% identical to any of SEQ ID NOs: 5, 8, 10, 13, 17, 19, 25, 27, 28, and 29. In some embodiments, the nucleic acid molecule comprises a nucleic acid sequence that is at least 60% identical to any of SEQ ID NOs: 5, 8, 10, 13, 17, 19, 25, 27, 28, and 29. In some embodiments, the nucleic acid molecule comprises a nucleic acid sequence that is at least 70% identical to any of SEQ ID NOs: 5, 8, 10, 13, 17, 19, 25, 27, 28, and 29. In some embodiments, the nucleic acid molecule comprises a nucleic acid sequence that is at least 80% identical to any of SEQ ID NOs: 5, 8, 10, 13, 17, 19, 25, 27, 28, and 29. In some embodiments, the nucleic acid molecule comprises a nucleic acid sequence that is at least 90% identical to any of SEQ ID NOs: 5, 8, 10, 13, 17, 19, 25, 27, 28, and 29.

In accordance with embodiments, the nucleic acid molecules comprise a K_{D} for fentanyl, or a derivative or analog thereof, that is less than about 500 nM. In some embodiments, the nucleic acid molecules comprise a K_{D} for fentanyl, or a derivative or analog thereof, that is less than about 400 nM. In some embodiments, the nucleic acid molecules comprise a K_{D} for fentanyl, or a derivative or analog thereof, that is less than about 300 nM. In some embodiments, the nucleic acid molecules comprise a K_{D} for fentanyl, or a derivative or analog thereof, that is less than about 200 nM. In some embodiments, the nucleic acid molecules comprise a K_{D} for fentanyl, or a derivative or analog thereof, that is less than about 100 nM. In some embodiments, the nucleic acid molecules comprise a K_{D} for fentanyl, or a derivative or analog thereof, that is less than about 50 nM. In some embodiments, the nucleic acid molecules comprise a K_{D} for fentanyl, or a derivative or analog thereof, that is less than about 25 nM.

Embodiments of the present disclosure also include a single-stranded nucleic acid molecule capable of binding fentanyl, or a derivative or analog thereof, comprising a nucleic acid sequence that is at least 50% identical to the following: X₁X₂AATGGX₃AGCATTGGTX₄X₅X₆X₇X₈X₉X₁₀X₁₁X₁₂X₁₃X₁₄X₁₅X₁₆; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ A, T, C, or G; X₁₁ is A, T, C, or G; X₁₂ is A, T, C, or G; X₁₃ is A, T, C, or G; X₁₄ is A, T, C, or G; X₁₅ is A, T, C, or G; and X₁₁ is A, T, C, or G (SEQ ID NO: 3). In some embodiments, the nucleic acid sequence is at least 60% identical to the following: X₁X₂AATGGX₃AGCATTGGTX₄X₅X₆X₇X₈X₉X₁₀X₁₁X₁₂X₁₃X₁₄X₁₅X₁₆; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ A, T, C, or G; X₁₁ is A, T, C, or G; X₁₂ is A, T, C, or G; X₁₃ is A, T, C, or G; X₁₄ is A, T, C, or G; X₁₅ is A, T, C, or G; and X₁₁ is A, T, C, or G (SEQ ID NO: 3). In some embodiments, the nucleic acid sequence is at least 70% identical to the following: X₁X₂AATGGX₃AGCATTGGTX₄X₅X₆X₇X₈X₉X₁₀X₁₁X₁₂X₁₃X₁₄X₁₅X₁₆; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ A, T, C, or G; X₁₁ is A, T, C, or G; X₁₂ is A, T, C, or G; X₁₃ is A, T, C, or G; X₁₄ is A, T, C, or G; X₁₅ is A, T, C, or G; and X₁₁ is A, T, C, or G (SEQ ID NO: 3). In some embodiments, the nucleic acid sequence is at least 80% identical to the following: X₁X₂AATGGX₃AGCATTGGTX₄X₅X₆X₇X₈X₉X₁₀X₁₁X₁₂X₁₃X₁₄X₁₅X₁₆; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ A, T, C, or G; X₁₁ is A, T, C, or G; X₁₂ is A, T, C, or G; X₁₃ is A, T, C, or G; X₁₄ is A, T, C, or G; X₁₅ is A, T, C, or G; and X₁₁ is A, T, C, or G (SEQ ID NO: 3). In some embodiments, the nucleic acid sequence is at least 90% identical to the following: X₁X₂AATGGX₃AGCATTGGTX₄X₅X₆X₇X₈X₉X₁₀X₁₁X₁₂X₁₃X₁₄X₁₅X₁₆; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ A, T, C, or G; X₁₁ is A, T, C, or G; X₁₂ is A, T, C, or G; X₁₃ is A, T, C, or G; X₁₄ is A, T, C, or G; X₁₅ is A, T, C, or G; and X₁₁ is A, T, C, or G (SEQ ID NO: 3).

In some embodiments, X₁ is A or G; X₂ is A or G; X₃ is C or A, X₄ is G or T, X₅ is A or G; X₆ is C or G; X₇ is T or G; X₈ is G or T; X₉ is G or A; X₁₀ G or T; X₁₁ is T or G; X₁₂ is T or C; X₁₃ is G or T; X₁₄ is C or T; X₁₅ is T or C; and X₁₁ is T or C (SEQ ID NO: 4). In accordance with these embodiments, the nucleic acid molecule comprises a nucleic acid sequence that is at least 50% identical to any of SEQ ID NOs: 6, 9, 11, 15, and 26. In some embodiments, the nucleic acid molecule comprises a nucleic acid sequence that is at least 60% identical to any of SEQ ID NOs: 6, 9, 11, 15, and 26. In some embodiments, the nucleic acid molecule comprises a nucleic acid sequence that is at least 70% identical to any of SEQ ID NOs: 6, 9, 11, 15, and 26. In some embodiments, the nucleic acid molecule comprises a nucleic acid sequence that is at least 80% identical to any of SEQ ID NOs: 6, 9, 11, 15, and 26. In some embodiments, the nucleic acid molecule comprises a nucleic acid sequence that is at least 90% identical to any of SEQ ID NOs: 6, 9, 11, 15, and 26.

In accordance with these embodiments, the nucleic acid molecules comprise a K_{D} for fentanyl, or a derivative or analog thereof, that is less than about 500 nM. In some embodiments, the nucleic acid molecules comprise a K_{D} for fentanyl, or a derivative or analog thereof, that is less than about 400 nM. In some embodiments, the nucleic acid molecules comprise a K_{D} for fentanyl, or a derivative or analog thereof, that is less than about 300 nM. In some embodiments, the nucleic acid molecules comprise a K_{D} for fentanyl, or a derivative or analog thereof, that is less than about 200 nM. In some embodiments, the nucleic acid molecules comprise a K_{D} for fentanyl, or a derivative or analog thereof, that is less than about 100 nM. In some embodiments, the nucleic acid molecules comprise a K_{D} for fentanyl, or a derivative or analog thereof, that is less than about 50 nM. In some embodiments, the nucleic acid molecules comprise a K_{D} for fentanyl, or a derivative or analog thereof, that is less than about 25 nM.

In accordance with the above embodiments, the nucleic acid molecule is capable of binding fentanyl, or a derivative or analog thereof, under physiological conditions. In some embodiments, the nucleic acid molecule comprises a detection moiety. In some embodiments, the nucleic acid molecule is in solution or attached to a substrate.

In some embodiments, the fentanyl derivative or analog is selected from the group consisting of: acetyl fentanyl, furanyl fentanyl, acrylfentanyl, butyryl fentanyl, valeryl fentanyl, cyclopropyl fentanyl, methoxyacetyl fentanyl, p-fluoro fentanyl, o-methyl furanyl fentanyl, o-methoxy furanyl fentanyl, p-methoxy furanyl fentanyl, p-methoxy butyryl fentanyl, FIBF, alpha-methyl thiofentanyl, 3,4-dimethoxy fentanyl, beta-hydroxy thiofentanyl, cis-3-methyl fentanyl, trans-3-methyl fentanyl, cis-3-methyl thiofentanyl, trans-3-methyl thiofentanyl, sufentanil, alfentanil, remifentanil, benzylfentanyl, N-benzyl furanyl norfentanyl, N-benzyl m-fluoro norfentanyl, N-benzyl o-fluoro norfentanyl, N-benzyl phenyl norfentanyl, norfentanyl, and 4-ANPP.

Embodiments of the present disclosure also include a vector comprising any of the nucleic acid sequences of the aptamers described herein. In some embodiments, the vector is an expression vector.

### 3. Methods of Use

Embodiments of the present disclosure also include methods for detecting fentanyl, or a derivative or analog thereof. In accordance with these embodiments, one method includes combining any of the nucleic acid molecules of the aptamers described herein comprising a fluorescent moiety with a quencher-labeled nucleic acid molecule that is at least partially complementary to the nucleic acid molecules of the aptamers described herein to form a quenched composition, and exposing the quenched composition to a sample comprising or suspected of comprising fentanyl, or a derivative or analog thereof. In some embodiments, the presence of the fentanyl, or a derivative or analog thereof, in the sample displaces the quencher-labeled nucleic acid molecule, thereby producing a fluorescent signal proportional to the concentration of the fentanyl, or a derivative or analog thereof, in the sample.

In other embodiments, a method for detecting fentanyl, or a derivative or analog thereof, includes combining any of the nucleic acid molecules of the aptamers described herein with a reporter compound that binds to the nucleic acid molecules of the aptamers described herein to form a complexed composition, and exposing the complexed composition to a sample comprising or suspected of comprising fentanyl, or a derivative or analog thereof. In some embodiments, the presence of the fentanyl, or a derivative or analog thereof, in the sample displaces the reporter compound, thereby allowing the reporter compound to form detectable aggregates proportional to the concentration of the fentanyl, or a derivative or analog thereof, in the sample.

In other embodiments, a method for detecting fentanyl, or a derivative or analog thereof, includes immobilizing any of the nucleic acid molecules of the aptamers described herein to an electrically conductive substrate. In some embodiments, the nucleic acid molecules of the aptamers described herein comprise a redox tag, to form a detection sensor, and exposing the detection sensor to a sample comprising or suspected of comprising fentanyl, or a derivative or analog thereof. In some embodiments, the presence of the fentanyl, or a derivative or analog thereof, in the sample binds the nucleic acid molecules of the aptamers described herein thereby producing an electrochemical signal proportional to the concentration of the fentanyl, or a derivative or analog thereof, in the sample.

In accordance with these embodiments, the sample is a biological sample from a human subject. In some embodiments, the biological sample is a saliva sample, a urine sample, a blood sample, a serum sample, a plasma sample, a fecal sample, a CSF sample, or a tissue sample.

In some embodiments of these methods, the fentanyl derivative or analog is selected from the group consisting of: acetyl fentanyl, furanyl fentanyl, acrylfentanyl, butyryl fentanyl, valeryl fentanyl, cyclopropyl fentanyl, methoxyacetyl fentanyl, p-fluoro fentanyl, o-methyl furanyl fentanyl, o-methoxy furanyl fentanyl, p-methoxy furanyl fentanyl, p-methoxy butyryl fentanyl, FIBF, alpha-methyl thiofentanyl, 3,4-dimethoxy fentanyl, beta-hydroxy thiofentanyl, cis-3-methyl fentanyl, trans-3-methyl fentanyl, cis-3-methyl thiofentanyl, trans-3-methyl thiofentanyl, sufentanil, alfentanil, remifentanil, benzylfentanyl, N-benzyl furanyl norfentanyl, N-benzyl m-fluoro norfentanyl, N-benzyl o-fluoro norfentanyl, N-benzyl phenyl norfentanyl, norfentanyl, and 4-ANPP.

Embodiments of the present disclosure also include a method of identifying a nucleic acid aptamer capable of binding fentanyl, or a derivative or analog thereof. In accordance with these embodiments, the method includes (i) exposing a library of candidate aptamers to fentanyl, or a derivative or analog thereof; (ii) collecting candidate aptamers exhibiting rapid target association kinetics; and (iii) repeating steps (i) and (ii) for at least one addition round of selection to generate a plurality of high-affinity candidate aptamers.

In some embodiments, the method further comprises sequencing the plurality of high-affinity candidate aptamers. In some embodiments, the method further comprises characterizing the plurality of high-affinity candidate aptamers using isothermal titration calorimetry (ITC). In some embodiments, the method further comprises assessing binding specificity of the plurality of high-affinity candidate aptamers using an exonuclease-based fluorescence assay.

### 4. Materials and Methods

Chemicals and oligonucleotides. Exonuclease I (Exo I; E. coli) (20 U/μL) and Exonuclease III (Exo III; E. coli) (100 U/μL) were purchased from New England Biolabs. Alprazolam, (±)-threo-methylphenidate HCl, lorazepam, clonazepam, cis-tramadol HCl, (±)-methadone HCl, ethylone HCl polymorph B, fentanyl HCl, heroin HCl, oxycodone HCl, Δ⁹-tetrahydrocannabinol, AB-FUBINACA, (+)-methamphetamine HCl, and amphetamine HCl were purchased from Cayman Chemicals. Citalopram HBr and amoxapine were purchased from TCI chemicals. Clomipramine HCl, and lidocaine HCl monohydrate were purchased from Alfa Aesar. Chlorpromazine HCl, procaine HCl, quinine hemisulfate monohydrate, diphenhydramine HCl, benzocaine, cocaine HCl, (+)-pseudoephedrine HCl, naloxone HCl, and naltrexone HCl were purchased from Sigma Aldrich. Formamide was purchased from Thermo Fisher Scientific, and SYBR Gold was purchased from Invitrogen. 3 kDa cut-off spin filters were purchased from Millipore Sigma, and 800 μL micro-gravity columns were purchased from Bio-Rad. QIAquick PCR purification kits were purchased from Qiagen. GoTaq Hot Start Colorless Master Mix was purchased from Promega. Human serum (normal pool), Nunc 384-well black plates, and streptavidin-modified agarose beads were purchased from Thermo Fisher Scientific. 10× phosphate buffered saline (PBS; 46-013-CM, molecular biology grade) and molecular biology grade water were purchased from Corning. Unless otherwise specified, all other chemicals were purchased from Sigma Aldrich. All modified and unmodified oligonucleotides were purchased from Integrated DNA technologies and dissolved in molecular biology grade water. The random DNA library and Cy5- and Iowa black RQ-modified oligonucleotides were purchased with HPLC purification and dissolved in 1×TE buffer (10 mM Tris-HCl with 1 mM EDTA, pH 8.0). 5′-thiolated and 3′-methylene blue-modified aptamers were purchased with dual HPLC purification and dissolved in 1×TE buffer. DNA sequences employed in this work are shown in Table 1 below.

TABLE 1
DNA sequences.

| | | SEQ ID |
|---|---|---|
| Sequences ID | Sequence (5′-3′) | NO: |
| DNA library | GGAGGCTCTCGGGACGAC(N30)GTCGTCCCGCCTTT | 30 |
| | AGGATTTACAG | |
| Bio-cDNA | GTCGTCCCGAGAGCCATA-/3BioTEG/ | 31 |
| Forward primer | GGAGGCTCTCGGGACGAC | 32 |
| Biotinylated-reverse | /5Biosg/-CTGTAAATCCTAAAGGCGGGACGAC | 33 |
| primer | | |
| Illumina-FP | ACACTCTTTCCCTACACGACGCTCTTCCGATCT(N4S) | 34 |
| | GGAGGCTCTCGGGACGAC | |
| Illumina-RP | GACTGGAGTTCAGACGTGTGCTCTTCCGATCT(N43)C | 35 |
| | TGTAAATCCTAAAGGCGGGACGAC | |
| E1 | GGGACGACTGCTTGGGTAGGTCGGGCTTGGGTTCGG | 5 |
| | CGGTCGTCCC | |
| E2 | GGGACGACAAAATGGCAGCATTGGTGACGGAGTTG | 6 |
| | CTTGTCGTCCC | |
| E3 | GGGACGACAGGTCGGTCAGTGGTAGGGTAGGTCTA | 7 |
| | TCTGTCGTCCC | |
| E4 | GGGACGACAGCTTGGTTTGGTCGGGGTAGGGTCCGG | 8 |
| | ATGTCGTCCC | |
| E5 | GGGACGACGTAATGGGAGCATTGGTCACGCAGGTG | 9 |
| | CACGTCGTCCC | |
| E6 | GGGACGACGGTTGGCATTGGGTCGGGTGTGGGTGC | 10 |
| | GGCGTCGTCCC | |
| E7 | GGGACGACAGAATGGCAGCATTGGTGAGTTATTTGC | 11 |
| | CTGTCGTCCC | |
| E8 | GGGACGACAAAAATGGAAGCATTGGTGACGGAGTT | 12 |
| | GCTTGTCGTCCC | |
| E9 | GGGACGACAGCTTGGTCTGGTCGGGGTAGGGTTCGG | 13 |
| | ATGTCGTCCC | |
| E10 | GGGACGACAAAAATGGAAGCATTGGTCCACGCAGT | 14 |
| | GTGCAGTCGTCCC | |
| L1 | GGGACGACAAAAATGGAAGCATTGGTTGTTGGTGCT | 15 |
| | TTGTCGTCCC | |
| L2 | GGGACGACGTGTAGGTGTGCACCCGCAGGGCTCTGC | 16 |
| | AGGTCGTCCC | |
| L3 | GGGACGACGGTTGGCTTGGGTCGGGTGTGGGTGCG | 17 |
| | GCGTCGTCCC | |
| L4 | GGGACGACAAAAATGGAAGCATTGGTTGTTGGTGC | 18 |
| | CTTGTCGTCCC | |
| L5 | GGGACGACAGCTTGGCTTGGTCGGGGTAGGGTTCGG | 19 |
| | ATGTCGTCCC | |
| L6 | GGGACGACAGCGTGGTTTGGTCGGGGTAGGGTTCG | 20 |
| | GATGTCGTCCC | |
| L7 | GGGACGACAAAAATGGAAGCATTGGTTGTCGGTGC | 21 |
| | TTTGTCGTCCC | |
| L8 | GGGACGACAAAAATGGAAGCATTGGTTGTTGGCGC | 22 |
| | TTTGTCGTCCC | |
| L9 | GGGACGACAGCTTGGTTTGGTTGGGGTAGGGTTCGG | 23 |
| | ATGTCGTCCC | |
| L10 | GGGACGACAAAAAATGGAAGCATTGGTTGTTGGTG | 24 |
| | CTTTGTCGTCCC | |
| P1 | GGGACGACAGCTTGGTTTGGTCGGGGTAGGGTTCGG | 25 |
| | ATGTCGTCCC | |
| P2 | GGGACGACTAATGGGAGCATTGGTCCACGCAGTGT | 26 |
| | GCAGTCGTCCC | |
| P3 | GGGACGACAATGGGTGGTGTTAGCGAGTGTCTTTCC | 27 |
| | TTGTCGTCCC | |
| P4 | GGGACGACGGTTGGCTTGGGGTCGGGTGTGGGTGC | 28 |
| | GGCGTCGTCCC | |
| P5 | GGGACGACTGCTTGGGTAGGTCGGGTTTGGGTTCGG | 29 |
| | CGGTCGTCCC | |
| E2-34 | ACAAAATGGCAGCATTGGTGACGGAGTTGCTTGT | 36 |
| L3-Cy5 | /5Cy5/TGGCTCTCGGGACGACGGTTGGCTTGGGTCGG | 37 |
| | GTGTGGGTGCGGCGTCGTCCC | |
| cDNA-IB | GTCGTCCCGAGAGC-/3IAbRQSp/ | 38 |
| E2-34-MB | /ThiolC6/ | 39 |
| | ACAAAATGGCAGCATTGGTGACGGAGTTGCTTGT- | |
| | /MB/ | |

N = random nucleotides; /5Biosg/ = biotin tag; /3BioTEG/ = biotin tag with extended triethylene glycol linker; /5Cy5/ = Cy5 fluorophore tag; /3IAbRQSp/ = Iowa Black RQ quencher tag; /ThiolC6/ = thiol group with six-carbon spacer; /MB/ = methylene blue redox tag.

Deionized (DI) water with a conductivity of 18.2 MΩ×cm was obtained using a Milli-Q EQ 7000 (Millipore Sigma). 3,3′-di(3-sulfopropyl)-4,5,4′,5′-dibenzo-9-methyl-thiacarbocyanine (MTC) was synthesized previously.

SELEX procedure. Isolation of fentanyl binding aptamers was done using the library-immobilized SELEX protocol with some modifications. The 1×SELEX buffer was prepared by diluting 5 mL of 10×PBS and 5 mL of 10 mM MgCl₂ to a final volume of 50 mL using molecular biology grade water. The final buffer and ion concentrations of 1×PBS (pH 7.4) are as follows: 10.10 mM Na₂HPO₄, 1.76 mM KH₂PO₄, 136.89 mM NaCl, 2.68 mM KCl, and 1 mM MgCl₂. The 73-nt library consisted of a random 30-nt domain flanked by two primer-binding sites. The initial library pool for the first round of selection comprised ~6×10¹⁴ oligonucleotides (1 nmol). The SELEX procedure consisted of library immobilization, washing with selection buffer or counter targets, elution of target-binding strands, library amplification, and finally, single-strand DNA generation. During SELEX, washing volumes and counter-target concentrations were gradually increased throughout selection, while the fentanyl concentration was gradually reduced. In each selection rounds, three target-eluted samples (fractions, 1, 2 and 3) were collected. For rounds 1-9, all fractions were combined at the end of each round and then PCR amplified and subjected to the next round of selection. For rounds 10-13, each fraction was independently PCR amplified and the target-binding affinity of each amplified pool was determined using a previously-reported gel-elution assay. Different target-eluted fractions were used for the next round of selection. For example, fraction 1 and fraction 2 from round 10 were combined and used as the input pool for round 11 selection. For rounds 12 and 13, only fraction 1 from the previous round was amplified and used as the input pool. Selection strategies and counter-selection conditions are detailed in Table 2 and Table 3 below.

TABLE 2
Selection strategy and conditions for isolating fentanyl-binding
aptamers via SELEX.

| | | Wash A | Counter | Wash B | Target |
|---|---|---|---|---|---|
| Round | Pool (pmol) | (times) | SELEX | (times) | (μM) |
| 1 | 1,000 | 10 | As described | NA | 100 |
| 2 | 350 | 20 | in Table 3 | 30 | 100 |
| 3 | 350 | 20 | | 30 | 100 |
| 4 | 300 | 40 | | 40 | 50 |
| 5 | 300 | 40 | | 40 | 50 |
| 6 | 300 | 40 | | 40 | 50 |
| 7 | 300 | 40 | | 40 | 50 |
| 8 | 300 | 40 | | 40 | 50 |
| 9 | 300 | 40 | | 40 | 50 |
| 10 | 300 | 40 | | 40 | 50 |
| 11 | 300 | 40 | | 40 | 10 |
| | (Fraction 1 & 2) | | | | |
| 12 | 300 | 40 | | 40 | 5 |
| | (Fraction 1) | | | | |
| 13 | 300 | 40 | | 40 | 5 |
| | (Fraction 1) | | | | |

Wash A and B were performed before and after counter SELEX, respectively. A 250 μl selection buffer was used for per wash.

TABLE 3
Selection condition for counter SELEX.

| R | Counter SELEX (# of total washes if >3) | | | | | | | | | | | |
|---|---|---|---|---|---|---|---|---|---|---|---|---|
| 1 | NA | | | | | | | | | | | |
| 2 | A | B | C | D | E | K | L | M | N | G1 | G2 | G3 |
| 3 | F | G | H | I | J | K | L | M | N | G1 | G2 | G3 |
| 4 | A (×6) | B(×6) | C(×6) | D | E | K | L | M | N | G1(×6) | G2 | G3 |
| 5 | F (×6) | G(×6) | H(×6) | I | J | K | L | M | N | G1(×6) | G2 | G3 |
| 6 | A (×12) | B(×12) | C(×12) | D | E | K | L | M | N | G1(×6) | G2 | G3 |
| 7 | F(×12) | G(×12) | H(×12) | I | J | K | L | M | N | G1(×6) | G2 | G3 |
| 8 | A(×12) | B(×12) | C(×12) | D | E | K | L | M | N | G1(×12) | G2 | G3 |
| 9 | F(×12) | G(×12) | H(×12) | I | J | K | L | M | N | G1(×12) | G2 | G3 |

| | | | | | | | | | | |
|---|---|---|---|---|---|---|---|---|---|---|
| 10 | A(×12) | B(×12) | C(×12) | D | E | K | L | M | N | O P Q R S T U V W |
| 11 | F(×12) | G(×12) | H(×12) | I | J | K | L | M | N | O P Q R S T U V W |

| | | | | | | | | | |
|---|---|---|---|---|---|---|---|---|---|
| 12 | M(×24) | D(×24) | A(×12) | B(×12) | C(×12) | E(×12) | F(×12) | G(×12) | H I J K L N O P Q R S T U V W |
| 13 | M(×24) | D(×24) | A(×12) | B(×12) | C(×12) | E(×12) | F(×12) | G(×12) | H I J K L N O P Q R S T U V W |

A = Clomipramine; B = Citalopram; C = Alprazolam; D = Methylphenidate; E = Methadone; F = Amoxapine; G = Lorazepam; H = Clonazepam; I = (+)-Amphetamine; J = Tramadol; K = Quinine; L = Chlorpromazine; M = Procaine; N = (+)-Methamphetamine; O = Diphenhydramine; P = Lidocaine; Q = Benzocaine; R = Cocaine; S = Heroin; T = Ethylone; U = Naloxone; V = Naltrexone; W = Oxycodone; G1 = Diphenhydramine, lidocaine, and benzocaine; G2 = Cocaine, heroin, and ethylone; G3 = Naloxone, naltrexone, and oxycodone.

All counter targets were prepared in 1×SELEX buffer, except clomipramine, citalopram, alprazolam, amoxapine, lorazepam, and clonazepam, which included 100 MeOH co-organic solvent. During rounds 2 and 3, all counter-target concentrations were 100 μM. From rounds 4-13, the concentration of each counter-target was increased to 250 μM, except for clomipramine, citalopram, alprazolam, amoxapine, lorazepam, and clonazepam which were kept at 100 μM. Each wash consisted of 250 μL of SELEX buffer. Each counter-target underwent three washes unless otherwise specified in the table. Between each counter-target, three washes with SELEX buffer were performed.

High-throughput sequencing (HTS). Fraction 1 and fraction 2 from round 10, and fraction 1 from round 11, round 12, and round 13 were submitted for high-throughput Illumina sequencing at GENEWIZ. Before submission, Illumina adapters were added to each pool via PCR using a customized forward and reverse primer (Table 1; Illumina-FP and Illumina-RP). Specifically, 10 nM of each pool was mixed with 1 μM Illumina FP and Illumina RP and subjected to 10 PCR cycles using the following conditions: 2 min at 95° C.; 9 cycles of 95° C. for 15 s, 58° C. for 30 s, and 72° C. for 45 s; and finally, 5 min at 72° C. PCR product size was confirmed using polyacrylamide gel electrophoresis, and the amplified product was purified using the QIAquick PCR purification kit and submitted for HTS. Prior to analysis, the sequence data of fraction 1 and fraction 2 from round 10 were combined for the purposes of calculating metrics such as enrichment fold. Primer sequences were trimmed from fasta files using cutadapt followed by further analysis using FASTAptamer to obtain sequence population data and assess enrichment between selection rounds and clustal omega to obtain sequence alignments.

Binding characterization using an exonuclease-based fluorescence assay. Binding profile of aptamer candidates was evaluated using a recently published exonuclease fluorescence assay. Specifically, 1 μL of aptamer (final concentration=0.5 μM) was added to 3.45 μL of Mg²⁺-free PBS (final 1×concentration=10.10 mM Na₂HPO₄, 1.76 mM KH₂PO₄, 136.89 mM NaCl, and 2.68 mM KCl, pH 7.4). The solution was heated to 95° C. for 10 min, immediately cooled on ice, and mixed with 0.5 μL MgCl₂ solution (final concentration=1 mM) and 0.05 μL BSA solution (final concentration=0.1 mg/mL). Next, 20 μL of buffer, fentanyl (final concentration 10 μM), or interferent solution (final concentrations: 10 μM for THC and AB-FUBINACA; 50 μM for clomipramine, citalopram, amoxapine, alprazolam, clonazepam, lorazepam; 250 μM for benzocaine, procaine, diphenhydramine, lidocaine, quinine, chlorpromazine, cocaine, (+)-methamphetamine, amphetamine, (+)-pseudoephedrine, ethylone, heroin, oxycodone, naloxone, naltrexone, methylphenidate, methadone, and cis-tramadol) was added and incubated at 25° C. for 30 min. Then, 25 μL of exonuclease mixture (final concentrations: 0.025 U/μL Exo III and 0.05 U/μL Exo I) was added and incubated at 25° C.; digestion progress was monitored by collecting 5 μL samples at various timepoints and mixing these with 30 μL of quench solution (final concentrations: 1.76 mM KH₂PO₄, 10.10 mM Na₂HPO₄, 136.89 mM NaCl, 2.68 mM KCl, 12.5% formamide, 10 mM EDTA, 1×SYBR Gold, pH 7.4) in the wells of a Nunc 384-well black plate. Fluorescence intensity of SYBR Gold (λₑₓ/λₑₘ=495/537 nm) was recorded using the Tecan Spark microplate reader. Enzymatic inhibition was calculated as resistance value (Rᵥₐₗᵤₑ). Specifically, the area-under-the-curve (AUC) of the digestion time-course was calculated in the absence and presence of the target, with Rᵥₐₗᵤₑ calculated as (AUC_{target}/AUC_{blank})−1.

Determination of aptamer binding affinity using isothermal titration calorimetry (ITC). All ITC experiments were performed at 23° C. in 1×PBS buffer using a MicroCal ITC200 instrument (Malvern). For each experiment, the aptamer dissolved in buffer was heated at 95° C. for 10 min and then immediately cooled on ice, followed by the addition of MgCl₂. The sample cell was loaded with 300 μL of aptamer solution. Fentanyl was dissolved in 1×PBS buffer and loaded in the syringe. The concentrations of aptamer and fentanyl used for each experiment are listed below in Table 4.

TABLE 4
Aptamer dissociation constants (KD), and ITC experiment conditions.

| Aptamer | Fentanyl (μM) | Aptamer (μM) | KD (nM) |
|---|---|---|---|
| E1 | 100 | 10 | 79 ± 7 |
| E2 | 200 | 20 | 143 ± 13 |
| E3 | 360 | 20 | 1,953 ± 173 |
| E4 | 200 | 20 | 257 ± 16 |
| E5 | 250 | 20 | 369 ± 22 |
| E6 | 200 | 20 | 58 ± 4 |
| E7 | 200 | 20 | 429 ± 36 |
| E8 | 500 | 50 | N/A |
| E9 | 200 | 20 | 192 ± 8 |
| E10 | 2000 | 80 | N/A |
| L1 | 200 | 20 | 426 ± 20 |
| L2 | 300 | 20 | 1,414 ± 83 |
| L3 | 70 | 7 | 25 ± 4 |
| L4 | 2,000 × 2 | 40 | 160,000 ± 6,000 |
| L5 | 200 | 20 | 121 ± 6 |
| L6 | 1,000 × 2 | 70 | 8,696 ± 346 |
| L7 | 300 | 20 | 1,350 ± 79 |
| L8 | 2000 | 80 | N/A |
| L9 | 2,000 × 2 | 40 | 80,000 ± 1,000 |
| L10 | 2,000 | 40 | 12,000 ± 3,000 |
| P1 | 200 | 20 | 97 ± 6 |
| P2 | 400 | 40 | 268 ± 8 |
| P3 | 400 | 40 | 666 ± 31 |
| P4 | 200 | 20 | 49 ± 3 |
| P5 | 200 | 20 | 128 ± 5 |

Typically, each titration began with a 0.4 μL purge injection, followed by 19 injections of 2 μL with 180-second spacing between injections. The spacing between injections and number of injections was altered for aptamers E10, L4, L5, L6, L8, L9, L10, P1, and P4 as necessary. Correction was performed on the raw data to account for dilution heat. The corrected data was analyzed using Origin 7 with the MicroCal analysis kit and fitted using a single-site binding model.

Detection of fentanyl in serum using the strand-displacement fluorescence assay. Detection of fentanyl in 50% serum was achieved using a previously reported strand-displacement fluorescence assay with Cy5-modified L3 aptamer (L3-Cy5) and Iowa black RQ-modified 14-nt cDNA (cDNA-IB). First, L3-Cy5 (final concentration=50 nM) was hybridized with various concentrations of cDNA-IB to identify the optimal ratio between aptamer and cDNA. Specifically, 72 μL of L3-Cy5 was mixed with 8 μL of cDNA-IB in 1×PBS buffer at various final concentrations (0, 15, 25, 31, 50, 62.5, 100, 125, 250, 500, or 1,000 nM). The solution was heated to 90° C. for 10 min, and slowly cooled to room temperature over 20 min to promote annealing. Then, 70 μL of the solution was loaded into the wells of a Nunc 384-well black plate and the fluorescence intensity was immediately recorded using a Tecan Spark microplate reader with an excitation wavelength of 648 nm and emission wavelength of 685 nm. An optimal cDNA concentration of 62.5 nM was identified to achieve quenching efficiency of at least 80%. Under these optimized conditions, 60 μL of L3-Cy5-cDNA-IB complex (final concentrations 50 nM L3-Cy5 and 62.5 nM cDNA-IB) in 1×PBS buffer was subjected to the heating and cooling procedure described earlier. Then, 20 μL of 1×PBS buffer or fentanyl at various final concentrations in 1×PBS buffer (1, 2.5, 5, 10, 25, 50, 100, 250, 500, 1,000, 2,500, 5,000, 10,000, or 25,000 nM) was added and incubated for 30 min at room temperature. Finally, 70 μL of the mixture was loaded into wells of a Nunc 384-well black plate and the fluorescence intensity was recorded as described above. For fentanyl detection in 25% serum, 52 μL of L3-Cy5-cDNA-IB complex was prepared under the same conditions described above. 8 μL of fentanyl was spiked into 20 μL of 100% human serum to achieve various final concentrations (1, 2.5, 5, 10, 25, 50, 100, 250, 500, 1,000, 2,500, 5,000, 10,000, or 25,000 nM). This spiked serum was then added to the L3-Cy5-cDNA-IB complex and incubated for 30 min at room temperature. For fentanyl detection in 50% human serum, a similar procedure was performed, except 32 μL of L3-Cy5-cDNA-IB complex was initially prepared and then 40 μL of 100% human serum spiked with 8 μL of fentanyl was added to achieve the same range of final concentrations, and this was incubated for 30 min at room temperature. 70 μL of each mixture was then loaded into wells of a Nunc 384-well black plate and the fluorescence intensity was recorded.

Optimization of aptamer concentration for the dye-displacement assay. Various P2 concentrations were tested with a constant concentration of MTC dye to achieve optimal sensor performance as previously described. First, 40 μL of various concentrations of P2 (final concentration=0, 2, 4, 6, 8, 10, 12, 16, and 20 μM) in 1.01×PBS buffer was diluted in 158 μL of 1.01×PBS buffer containing SDS (final concentration=0.01% w/v) and allowed to sit at room temperature for 5 min. 2 μL of MTC dissolved in DMSO (final concentration 1.5 μM) was added directly to each solution and rapidly mixed. 72 μL of this mixture was then loaded into the wells of a transparent 384-well microplate, and the absorbance spectra were recorded from 400 to 800 nm with a 2-nm step size using a Tecan Spark microplate reader.

Detection of fentanyl using a dye-displacement assay. Fentanyl detection was performed using 1.5 μM MTC/8 μM P2. Specifically, a 39.2 μL solution of P2 was prepared by dissolving the aptamer in 0.505×PBS buffer containing SDS (final concentration=0.005% w/v). The solution was allowed to incubate at room temperature for 5 min. Then, 0.8 μL of MTC was added to the aptamer solution and rapidly mixed. Finally, 40 μL of various concentrations of fentanyl (final concentration=0, 0.1, 0.2, 0.4, 0.8, 1.6, 3.2, 6.4, 12.8, 25.6, or 51.2 μM) dissolved in 0.5×PBS buffer containing SDS (final concentration=0.005% w/v) was added to the aptamer-dye solution. 72 μL of each sample was then loaded into the wells of a transparent 384-well microplate, and the absorbance spectra were recorded from 400 to 800 nm with a 2-nm step size using a Tecan Spark microplate reader. Fentanyl detection in saliva was performed as described above with minor modifications. Specifically, 39.2 μL of P2 dissolved in 1.01×PBS containing SDS (final concentration=0.005% w/v) was allowed to incubate for 5 min followed by the addition of 0.8 μL of MTC with rapid mixing. Then, various concentrations of 4 μL fentanyl was spiked into 40 μL of saliva such that the final fentanyl concentrations were 0, 0.1, 0.2, 0.4, 0.8, 1.6, 3.2, 6.4, 12.8, 25.6, and 51.2 μM. Finally, 36 μL of the aptamer-dye mixture was added to 44 μL drug-spiked saliva. 72 μL of this mixture solution was then loaded into the wells of a transparent 384-well microplate, and absorbance measurements were performed as described above.

Fabrication of electrochemical aptamer-based (E-AB) sensors. E-AB sensors were fabricated using a 5′-thiolated, 3′-methylene blue-modified E2-34 aptamer (E2-34-MB) with a target-assisted immobilization strategy. First, the gold disk electrodes (2-mm diameter, CHI Instruments) were polished and electrochemically cleaned using a previously reported method. To prepare the aptamer for electrode modification, the disulfide group on the E2-34-MB was reduced by incubating in 100 mM Tris(2-carboxyethyl)phosphine hydrochloride solution for 2 h at room temperature in the dark. The freshly reduced aptamer was diluted into various concentrations in 1×PBS containing 50 μM fentanyl. The cleaned electrodes were rinsed with distilled water, dried with nitrogen, and immediately immersed in a solution of aptamer-target complex overnight in the dark at room temperature. The aptamer-modified electrodes were then backfilled with 1 mM 6-mercapto-1-hexanol in 1×PBS containing 50 μM fentanyl for 2 h at room temperature. Finally, the E2-34-MB-modified electrodes were thoroughly washed with deionized water and stored in 1×PBS before use.

Optimization of E-AB sensor performance. All electrochemical measurements were performed using the CHI760D electrochemical workstation and a three-electrode system including an E2-34-MB-modified gold working electrode, a platinum counter electrode (CHI Instruments), and an Ag/AgCl reference electrode (CHI Instruments). Square-wave voltammetry (SWV) was utilized for the measurements, and the signal gains were calculated using the equation: (I_{T}−I₀)/I₀×100%, in which I_{T} and I₀ represent the peak currents in the presence and absence of target, respectively. The square-wave measurement frequency was first optimized using the modified electrode in the range of 25-600 Hz in the absence and presence of 25 μM fentanyl. To investigate the effect of aptamer surface coverage on sensor signal, gold working electrodes were fabricated with various concentrations of E2-34-MB (50, 100, or 200 nM) and their surface coverage was measured using the Tarlov method. Sensor performance was evaluated by testing 0.1, 1, and 25 μM of fentanyl with a frequency of 200 Hz.

Detection of fentanyl in buffer, 50% urine, 50% saliva, and 50% serum and specificity testing. For fentanyl detection in buffer, the E2-34-MB fabricated E-AB sensors were challenged with fentanyl in the concentration range of 0-50 μM in 1×PBS. To perform fentanyl detection in biosamples, the urine and saliva pools were first collected using a previously reported protocol. 50% urine, saliva, or serum were prepared by mixing 2×PBS with pooled human urine, saliva, or serum at a 1:1 ratio (v/v). Fentanyl-spiked biosamples were prepared by spiking different concentrations of fentanyl into the diluted urine, saliva, or serum. For specificity testing, most interferents were prepared in 1×PBS with a final concentration of 100 μM. However, clomipramine, citalopram, alprazolam, amoxapine, lorazepam, and clonazepam were prepared in 1×PBS containing 1% methanol with a final concentration of 50 μM due to solubility limitations. SWV spectra were recorded for all samples, and the peak current was used to calculate signal gain. Cross-reactivities of the interferents were calculated against the signal gain from 10 μM fentanyl. All experimental data were reported as the average results from three independent working electrodes. Error bars represent the standard deviation for two working electrodes for each measurement.

Sequences. The various embodiments of the present disclosure include polynucleotides having the following nucleic acid sequences.

E1 Family Consensus Sequence (SEQ ID NO: 1): X₁GCTTGGX₂TAGGTCGGGX₃TTGGGTX₄CGGX₅X₆; wherein X₁ is A, T, C, or G; X₂ is A, T, C, or G; X₃ is A, T, C, or G; X₄ is A, T, C, or G; X₅ is A, T, C, or G; and X₆ is A, T, C, or G.

E1 Family Consensus Sequence (SEQ ID NO: 2): X₁GCTTGGX₂TAGGTCGGGX₃TTGGGTX₄CGGX₅X₆; wherein X₁ is T or A; X₂ is G or T; X₃ is T or C, X₄ is T or G, X₅ is C or A; and X₆ is G or T.

E2 Family Consensus Sequence (SEQ ID NO: 3): X₁X₂AATGGX₃AGCATTGGTX₄X₅X₆X₇X₈X₉X₁₀X₁₁X₁₂X₁₃X₁₄X₁₅X₁₆; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁ A, T, C, or G; X₁₁ is A, T, C, or G; X₁₂ is A, T, C, or G; X₁₃ is A, T, C, or G; X₁₄ is A, T, C, or G; X₁₅ is A, T, C, or G; and X₁₆ is A, T, C, or G.

E2 Family Consensus Sequence (SEQ ID NO: 4): X₁X₂AATGGX₃AGCATTGGTX₄X₅X₆X₇X₈X₉X₁₀X₁₁X₁₂X₁₃X₁₄X₁₅X₁₆; wherein X₁ is A or G; X₂ is A or G; X₃ is C or A, X₄ is G or T, X₅ is A or G; X₆ is C or G; X₇ is T or G; X₈ is G or T; X₉ is G or A; X₁₀ G or T; X₁₁ is T or G; X₁₂ is T or C; X₁₃ is G or T; X₁₄ is C or T; X₁₅ is T or C; and X₁₁ is T or C.

TABLE 5
Aptamer sequences.

| ID | Category | Sequence (5′-3′) | SEQ ID NO: |
|---|---|---|---|
| E1 | I | GGGACGACTGCTTGGGTAGGTCGGGCTTGGGTTCGGCGGTCGTCCC | 5 |
| E2 | I | GGGACGACAAAATGGCAGCATTGGTGACGGAGTTGCTTGTCGTCCC | 6 |
| E3 | I | GGGACGACAGGTCGGTCAGTGGTAGGGTAGGTCTATCTGTCGTCCC | 7 |
| E4 | I | GGGACGACAGCTTGGTTTGGTCGGGGTAGGGTCCGGATGTCGTCCC | 8 |
| E5 | I | GGGACGACGTAATGGGAGCATTGGTCACGCAGGTGCACGTCGTCCC | 9 |
| E6 | I | GGGACGACGGTTGGCATTGGGTCGGGTGTGGGTGCGGCGTCGTCCC | 10 |
| E7 | I | GGGACGACAGAATGGCAGCATTGGTGAGTTATTTGCCTGTCGTCCC | 11 |
| E8 | I | GGGACGACAAAAATGGAAGCATTGGTGACGGAGTTGCTTGTCGTCCC | 12 |
| E9 | I | GGGACGACAGCTTGGTCTGGTCGGGGTAGGGTTCGGATGTCGTCCC | 13 |
| E10 | I | GGGACGACAAAAATGGAAGCATTGGTCCACGCAGTGTGCAGTCGTCCC | 14 |
| L1 | II | GGGACGACAAAAATGGAAGCATTGGTTGTTGGTGCTTTGTCGTCCC | 15 |
| L2 | II | GGGACGACGTGTAGGTGTGCACCCGCAGGGCTCTGCAGGTCGTCCC | 16 |
| L3 | II | GGGACGACGGTTGGCTTGGGTCGGGTGTGGGTGCGGCGTCGTCCC | 17 |
| L4 | II | GGGACGACAAAAATGGAAGCATTGGTTGTTGGTGCCTTGTCGTCCC | 18 |
| L5 | II | GGGACGACAGCTTGGCTTGGTCGGGGTAGGGTTCGGATGTCGTCCC | 19 |
| L6 | II | GGGACGACAGCGTGGTTTGGTCGGGGTAGGGTTCGGATGTCGTCCC | 20 |
| L7 | II | GGGACGACAAAAATGGAAGCATTGGTTGTCGGTGCTTTGTCGTCCC | 21 |
| L8 | II | GGGACGACAAAAATGGAAGCATTGGTTGTTGGCGCTTTGTCGTCCC | 22 |
| L9 | II | GGGACGACAGCTTGGTTTGGTTGGGGTAGGGTTCGGATGTCGTCCC | 23 |
| L10 | II | GGGACGACAAAAAATGGAAGCATTGGTTGTTGGTGCTTTGTCGTCCC | 24 |
| P1 | III | GGGACGACAGCTTGGTTTGGTCGGGGTAGGGTTCGGATGTCGTCCC | 25 |
| P2 | III | GGGACGACTAATGGGAGCATTGGTCCACGCAGTGTGCAGTCGTCCC | 26 |
| P3 | III | GGGACGACAATGGGTGGTGTTAGCGAGTGTCTTTCCTTGTCGTCCC | 27 |
| P4 | III | GGGACGACGGTTGGCTTGGGGTCGGGTGTGGGTGCGGCGTCGTCCC | 28 |
| P5 | III | GGGACGACTGCTTGGGTAGGTCGGGTTTGGGTTCGGCGGTCGTCCC | 29 |

### 5. Examples

The accompanying Examples are offered as illustrative as a partial scope and particular embodiments of the disclosure and are not meant to be limiting of the scope of the disclosure.

The present disclosure describes the development of several sensitive and specific aptamer-based sensors for the rapid detection of fentanyl in various biological samples. Given that the concentration of fentanyl is typically quite low in these matrices, successful detection of this drug required aptamers with low nanomolar affinity. As described herein, a new library-immobilized SELEX method termed Kinetic-SELEX was developed that utilizes aptamer-target binding kinetics as a form of selection pressure, alongside thermodynamic pressure in the form of round-by-round reductions in target concentration. This method was created based on the finding that aptamer pools collected at various times after the target elution step displayed differing binding affinities, with the first fraction having the highest affinity for the target presumably due to their fast association kinetics. As such, SELEX was first performed using traditional protocols for several rounds to enrich binders to fentanyl, and then in later SELEX rounds, kinetic pressure was introduced by enriching aptamer pools collected from the first target eluent fraction to preferentially enrich high affinity aptamers. This Kinetic-SELEX approach, which focuses on enriching aptamers with high kₒₙ, differs from a selection method reported by others utilizing the volume dilution challenge to separate rapidly dissociating target binders from aptamers with low k_{off}.

Following SELEX, HTS analysis was utilized to identify aptamer candidates. Typically, aptamers are chosen based on their population in the final selection round, or their enrichment between rounds. As an alternative, candidates were selected based on their population growth rate throughout multiple rounds of SELEX. It was determined that relatively abundant aptamers (>1% population) that peaked in population in the final few rounds had the best binding affinity, followed by those still undergoing exponential growth, whereas aptamers with linear growth rates exhibited the poorest affinity. This outcome suggests that superior aptamers undergo rapid population growth during early rounds, while mediocre aptamers either fail to grow in population or enrich slowly.

After characterizing the fentanyl-binding properties of top aptamer candidates, the aptamer with the highest target-binding affinity, L3, (K_{D}=25±4) was used to develop a strand-displacement fluorescence assay that demonstrated excellent sensing performance, with a LOD of 2.5 nM fentanyl in 25% serum and 5 nM in 50% serum. This assay could be amenable for high-throughput detection of fentanyl in biological samples using a microplate reader. The specificity of ten aptamer candidates with nanomolar affinity for fentanyl were evaluated against all of the interferents employed in a counter-SELEX screen using an exonuclease-based fluorescence assay, and a structurally-related family of aptamers were identified (L1, E2 and P2) that exhibited superior specificity toward all tested interferents. Aptamer P2 was used to develop a dye-displacement colorimetric assay with the cyanine dye MTC, and this assay could detect fentanyl concentrations as low as 100 nM in 50% saliva, which is suitable for use in at-home or on-site drug screening.

Additionally, structure-switching functionality was introduced into the high-specificity aptamer, E2, producing E2-34, which was then modified with a 5′-thiol and 3′-methylene blue redox tag (termed E2-34-MB). E2-34-MB was used to fabricate an E-AB sensor. This sensor produced a robust signal-to-noise ratio with a LOD of 10 nM fentanyl in 50% human urine, serum, and saliva, and minimal response to a wide range of non-target interferents. This E-AB sensor could potentially be employed to assess fentanyl overdose in a variety of settings, including ambulances or hospital emergency rooms. By converting the current E-AB sensor to a paper-based device, as demonstrated previously, the cost of testing could be lowered without compromising on analytical performance.

### Example 1

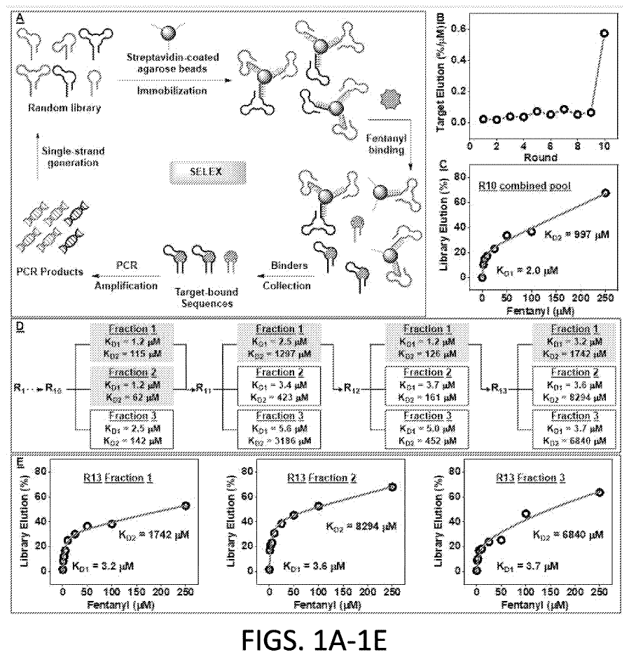

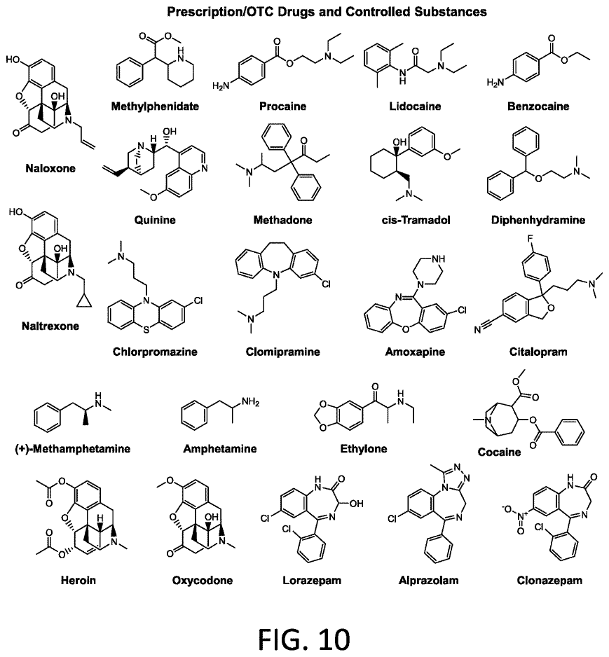

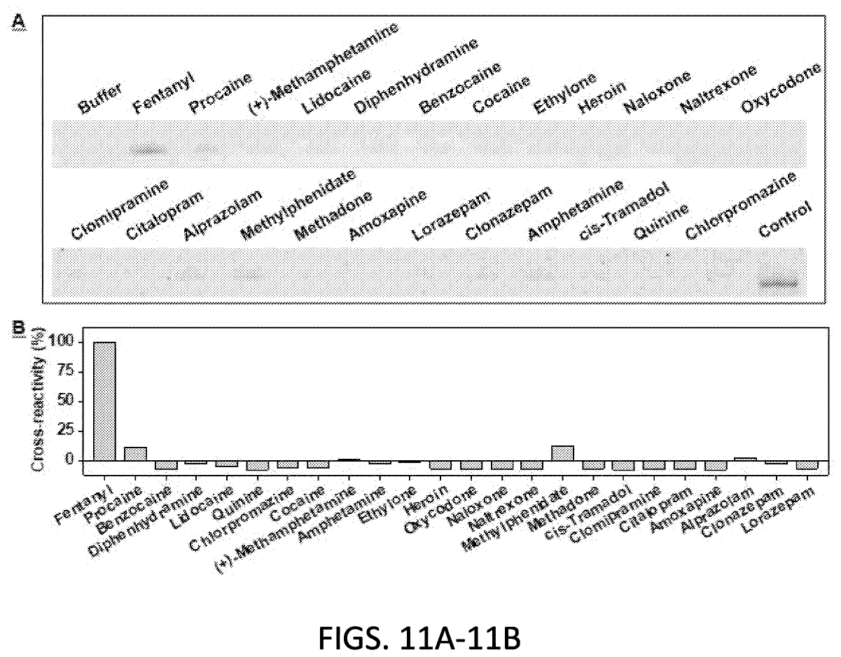

Initial SELEX rounds for isolation of fentanyl-binding aptamers. Library-immobilized SELEX was performed with a 73-nucleotide (nt) stem-loop-structured DNA library (Table 1) using fentanyl as the target. Each oligonucleotide in the library contains a 30-nt random region that serves as the putative binding domain, an 8-base-pair (bp) stem, and two primer-binding sites at both termini for PCR amplification. The library sequences also contained a fixed-sequence region that can hybridize with a biotinylated complementary 15-nt DNA (cDNA) to enable library immobilization on streptavidin-coated agarose beads. When the target is added, library strands that bind to the target undergo a conformational change and are released into solution. These sequences are then collected and amplified for another round of selection (FIG. 1A). To isolate aptamers that bind to fentanyl optimally in serum, SELEX was performed in 1×PBS (pH 7.4), which mimics biological pH and ionic strength. Details on the employed selection conditions and strategies are provided in Table 2 and Table 3. Specifically, experiments were conducted using 1 nmol of library (10¹⁴ unique sequences) and 100 μM fentanyl to elute target binders in the first round of selection. For the second and third rounds, 350 pmol library and 100 μM fentanyl were used, with counter-SELEX against a variety of interferent compounds (FIG. 10) at concentrations ranging between 100-250 μM. These interferents included cutting agents and adulterants (e.g., procaine, lidocaine, quinine, benzocaine, diphenhydramine, and chlorpromazine), prescription medications (e.g., methadone, cis-tramadol, amoxapine, clomipramine, methylphenidate, naloxone, naltrexone, citalopram, oxycodone, lorazepam, alprazolam, and clonazepam), and illicit substances (e.g., amphetamine, cocaine, (+)-methamphetamine, heroin, and ethylone). Most counter-targets were used individually, while others were employed in groups (Table 3). To increase stringency, the concentration of fentanyl was lowered to 50 μM in the fourth round. Until round 10, the amount of pool eluted by fentanyl remained consistent at around 0.7-2.6%. This is in contrast to the relatively large amount of pool eluted by certain counter-targets, such as clomipramine, alprazolam, clonazepam, citalopram, diphenhydramine, lidocaine, and benzocaine. In the tenth round, the proportion of fentanyl-eluted pool dramatically increased to 8.6%, indicating that the pool was enriched with aptamers that bound to this target (FIG. 1B), and pool elution by all counter-targets was greatly decreased, except for alprazolam which remained at ~6% (data not shown). A previously-reported gel elution assay was then used to determine the fentanyl-binding affinity and specificity of this round 10 pool. The round 10 pool demonstrated a distinct biphasic binding profile, with one population of sequences binding strongly to fentanyl with a dissociation constant (K_{D}) of 2 μM and another population with a much higher K_{D} of ~1 mM (FIG. 1C). Minimal cross-reactivity was observed to all counter-targets except for procaine and methylphenidate (~10%) (FIG. 11).

### Example 2

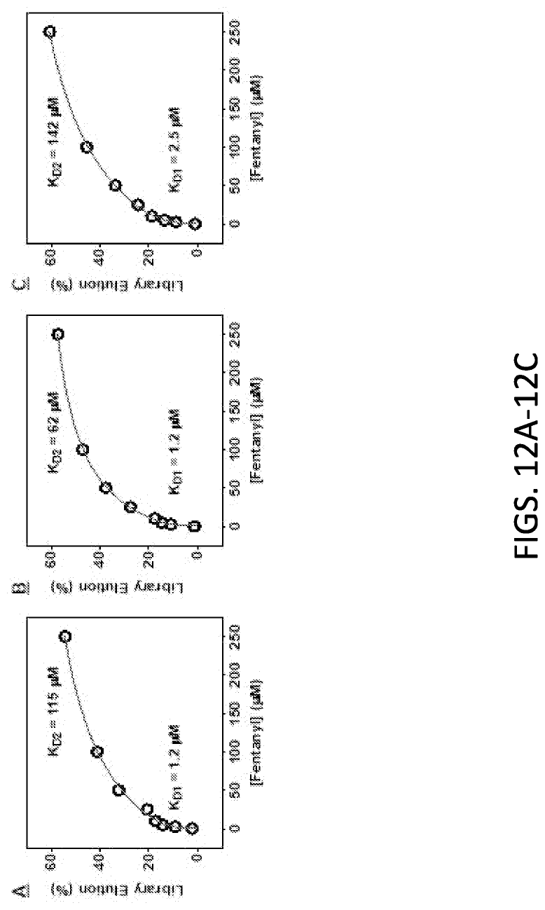

Using binding kinetics as selection pressure to isolate high affinity aptamers. The binding behavior of the round 10 pool indicated the pool consisted of low- and moderate-affinity binders (>40%) with a minority of high-affinity binders (<20%). This result was confounding because a relatively small amount of target (50 μM) had been used for the past six rounds. It was posited that high-affinity binders could be preferentially enriched by employing binding kinetics as an additional means to increase selection stringency. In conventional library-immobilized SELEX, targets are applied sequentially to the library-bead assembly in a gravity column as three individual 250 μL “fractions.” The library strands that elute in the first fraction (fraction 1) presumably either rapidly dissociate from their cDNA (high cDNA k_{off}) or have rapid target-binding kinetics (kₒₙ). Given that K_{D} is equal to k_{off}/kₒₙ, sequences with fast kₒₙ have the potential to offer high binding affinities. In the SELEX procedure, each aliquot takes ~3 min to elute from the column, and therefore the final eluted fraction (fraction 3) contains aptamers that require nearly 10 min to bind the target, fold, and displace from their cDNA. Logically, those sequences should have relatively a low cDNA k_{off} or slower kₒₙ, and neither of these binding properties confers high binding affinity. Therefore, it was hypothesized that aptamers collected in fraction 1 should have greater target-binding affinity than those in fraction 3. To test this hypothesis, fractions 1, 2, and 3 from round 10 were separately PCR amplified and then their fentanyl-binding affinities were characterized using the gel elution assay. As expected, the overall binding affinity of fraction 1 was greater than that of fraction 3. Each of the binding curves indicated the presence of two distinct populations—for fraction 1, one bound with K_{D1}=1.2 μM and the other with K_{D2}=115 μM. In contrast, fraction 3 had slightly poorer binding, with K_{D1}=2.5 μM and K_{D2}=142 μM. Fraction 2 had better overall affinity (K_{D1}=1.2 μM and K_{D2}=62 μM) than fraction 3 (FIG. 12). This result indicated that aptamers with poor binding affinity were primarily being eluted in the last target elution, and were therefore excluded amplicons from fraction 3 in subsequent rounds of SELEX.

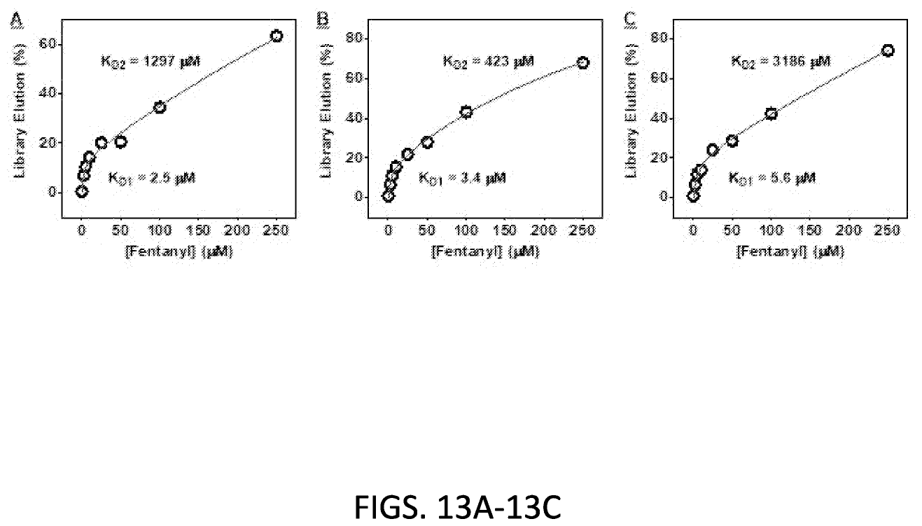

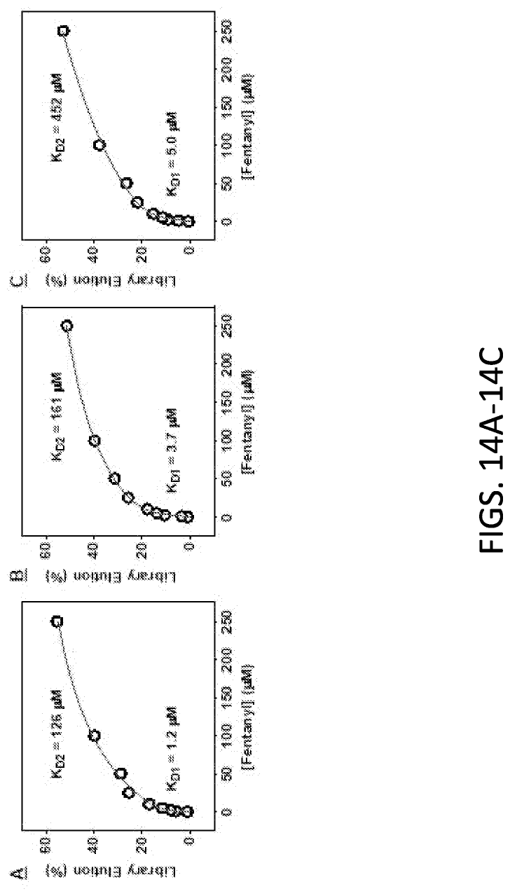

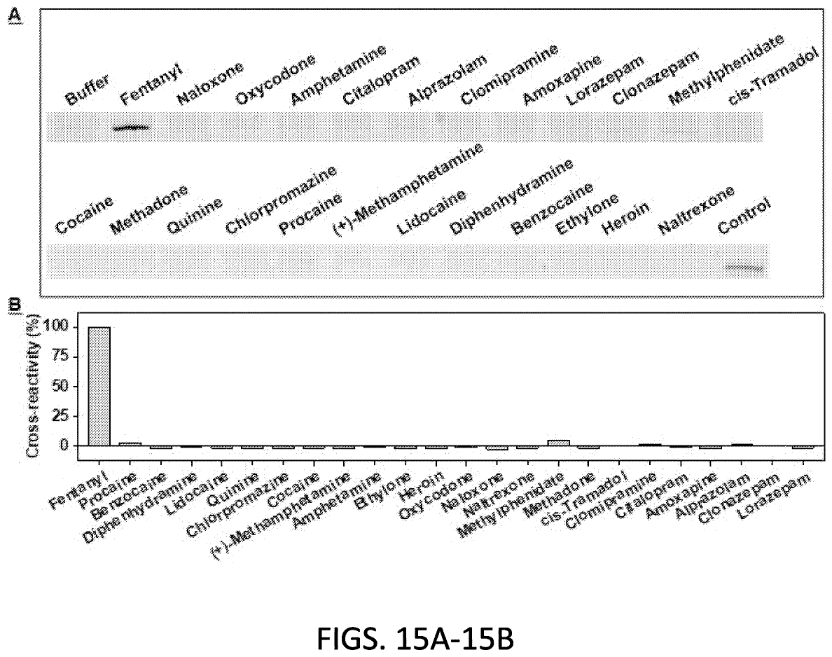

Therefore, a modified SELEX approach was performed, with the workflow from round 10-13 detailed in FIG. 1D. For the eleventh round, the PCR amplicons from fraction 1 and 2 of the tenth round were combined to perform SELEX, and the concentration of fentanyl was reduced to 10 μM to further enrich binders with high affinity. Despite the increased selection stringency, 8.5% of the pool was eluted overall after addition of the three fentanyl aliquots, with 1.2% of the pool eluted in the first fraction. Although relatively low, this elution was approximately 10-fold higher than that achieved with 250 μL buffer wash alone (0.1%). Most of the eluted sequences were in fractions 2 and 3, with target elution of 3.9% and 3.4%, respectively. Each target fraction was PCR amplified separately and the affinity of each of the resulting pools was determined as above. Again, all three pools exhibited biphasic binding curves indicative of a minority (~20%) high-affinity population and a majority low-affinity population (K_{D}>400 μM) (FIG. 13), and fraction 1 displayed better binding affinity (K_{D1}=2.5 μM) than the other fractions (K_{D1}=3.4 μM for fraction 2 and K_{D1}=5.6 μM for fraction 3). Therefore, for the twelfth round, only amplicons from the first fraction of round 11 were used, and the concentration of fentanyl was cut in half to 5 μM. The total target elution was 5%, and again biphasic binding curves were observed for each fraction, although the population of high-affinity binders rose to 25%, while the population of weak binders decreased from ~45% to ~35% (FIG. 14). Also, the affinity of fraction 1 was greater than that of the other two fractions. In the thirteenth round, the amplicons of fraction 1 from the previous round were used as the starting pool with 5 μM fentanyl, and observed 5.8% target elution. The gel-elution assay revealed that fraction 1 now primarily contained high-affinity binders (40% of the population, K_{D1}=3.2 M) with a much smaller population of mediocre binders (~10% of the pool). However, fractions 2 and 3 still contained a heterogenous group of both high- and low-affinity binders (FIG. 1E). The specificity of fraction 1 from round 13 was further assessed using the gel-elution assay, and <5% cross-reactivity against all counter-targets was observed (FIG. 15). It was therefore concluded that aptamers with robust affinity and specificity for fentanyl were obtained by this stage of SELEX.

### Example 3

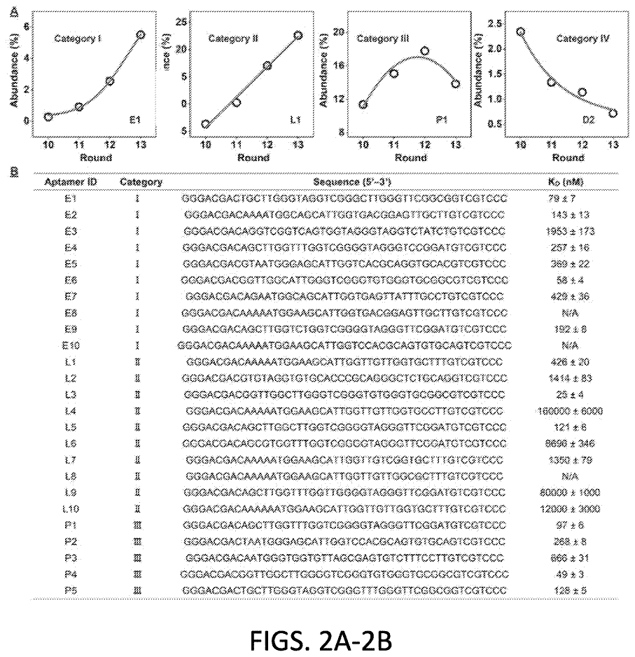

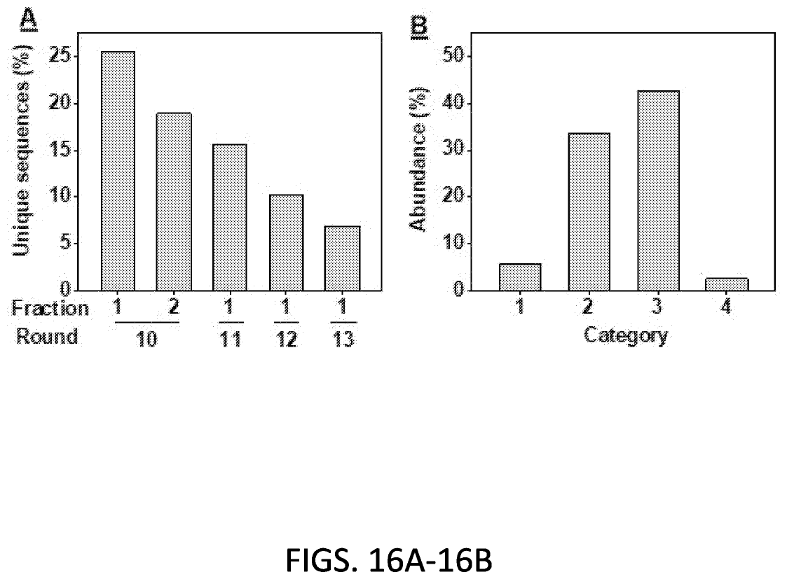

High-throughput sequencing (HTS) of SELEX pools. To identify aptamer candidates for sensor development, HTS analysis was performed of fractions 1 and 2 of round 10 and fraction 1 from round 11, 12, and 13. The overall diversity of the pools decreased as the rounds progressed, from 25.6% unique sequences in the tenth round to 6.9% in the thirteenth round (FIG. 16A). To select candidate aptamers, the population of each sequence was plotted over the course of these selection rounds and the sequences were sorted into four categories, including sequences: 1) with exponential growth rates, 2) with linear growth rate, 3) that had peaked in population, and 4) consistently decreasing in population (FIG. 2A). Sequences in category four or that did not demonstrate a clear growth pattern (typically those with <30 reads per million) were omitted from further analysis. The presumption was that the highest-affinity binders were those that underwent exponential growth (5.7% of sequences) or those that had high abundance (>10%) and peaked in population (33% of sequences) (FIG. 16B). The ten most abundant candidates were selected from category 1 and the five most abundant candidates were selected from category 3; the ten most abundant candidates from category 2 were selected as a control. Notably, the five category 3 candidates were also the most abundant aptamers in the round 13 pool, making up 33% of the total population.

### Example 4

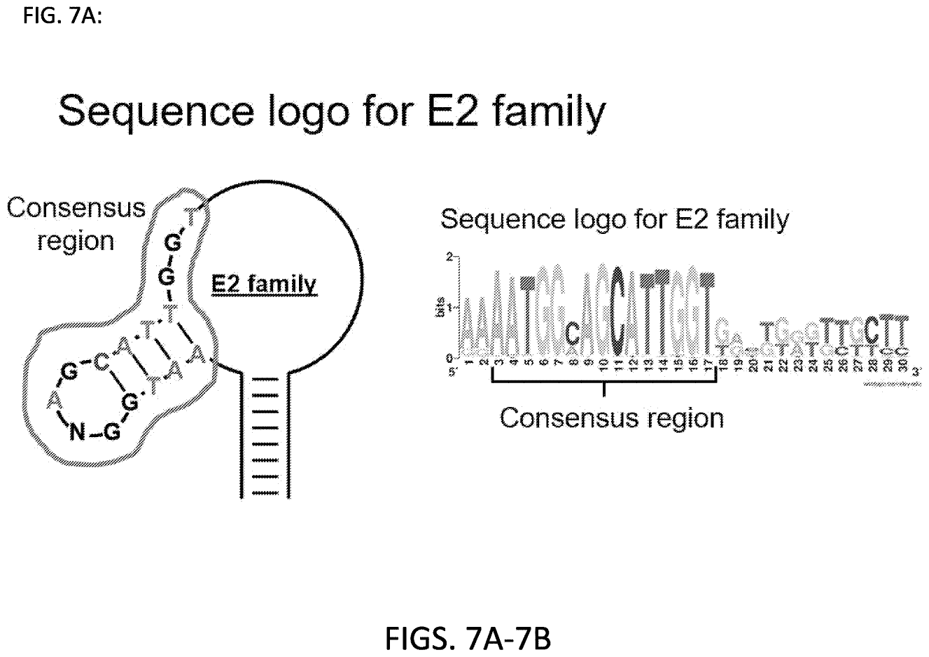

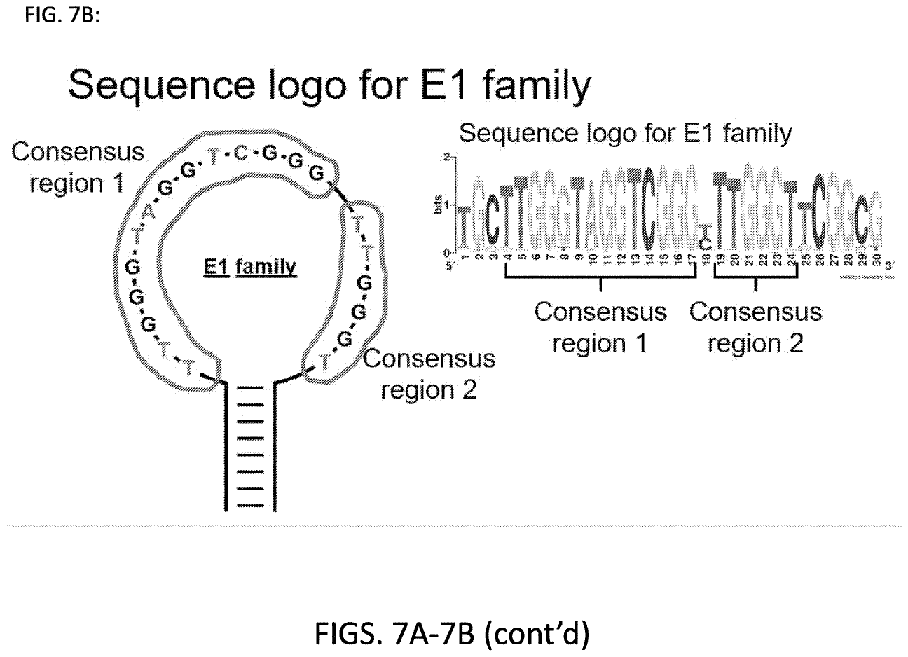

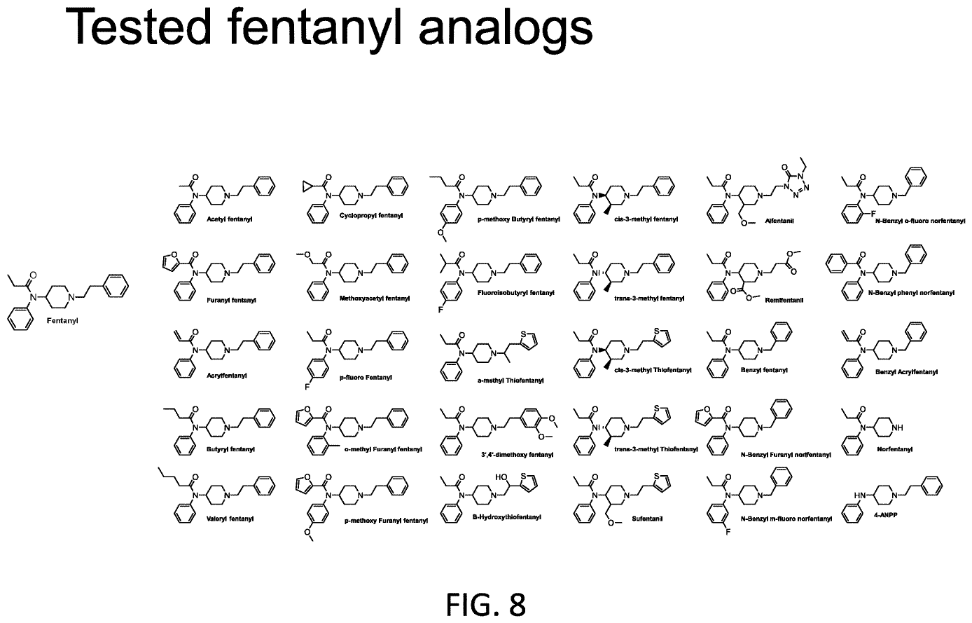

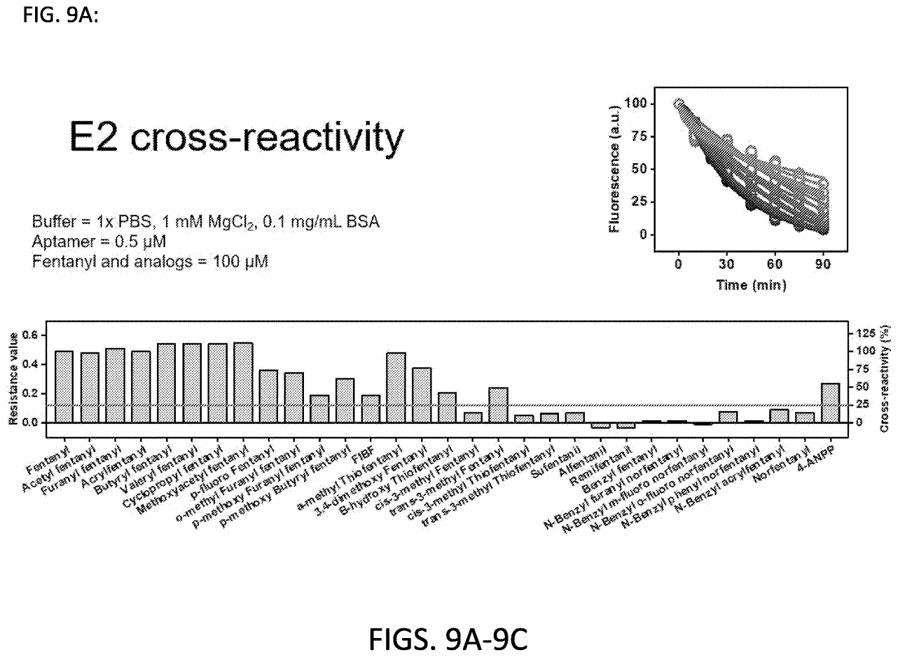

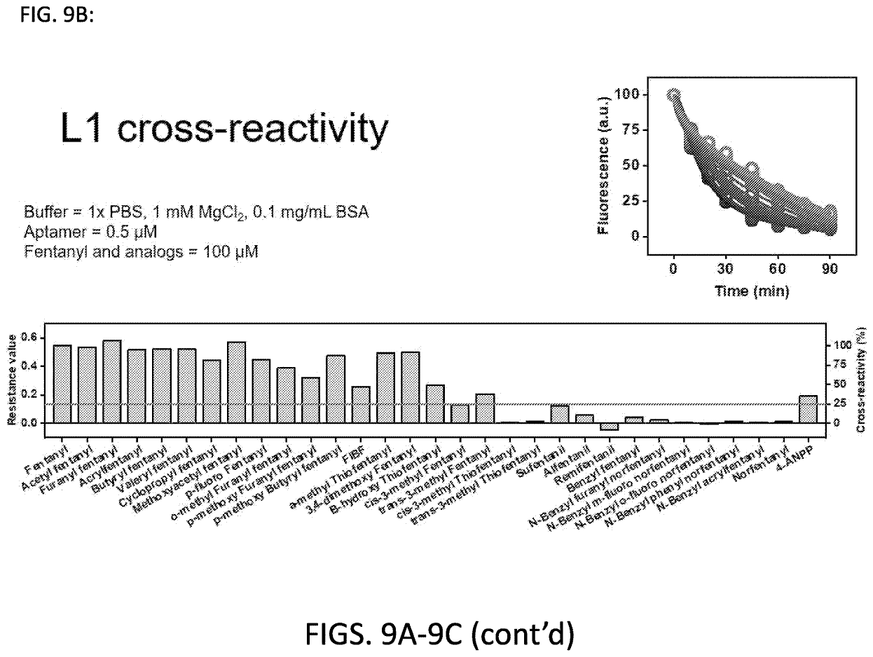

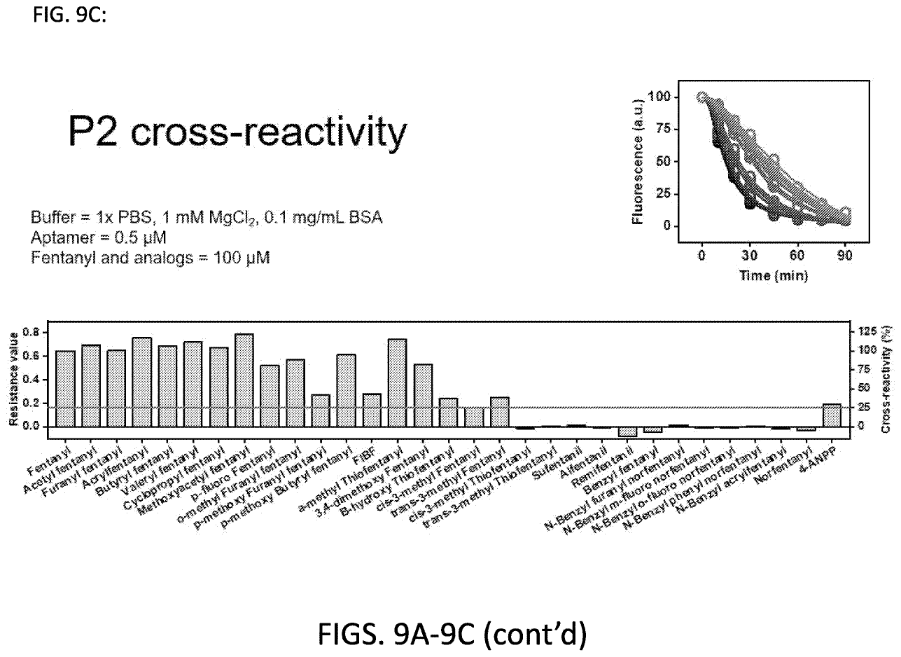

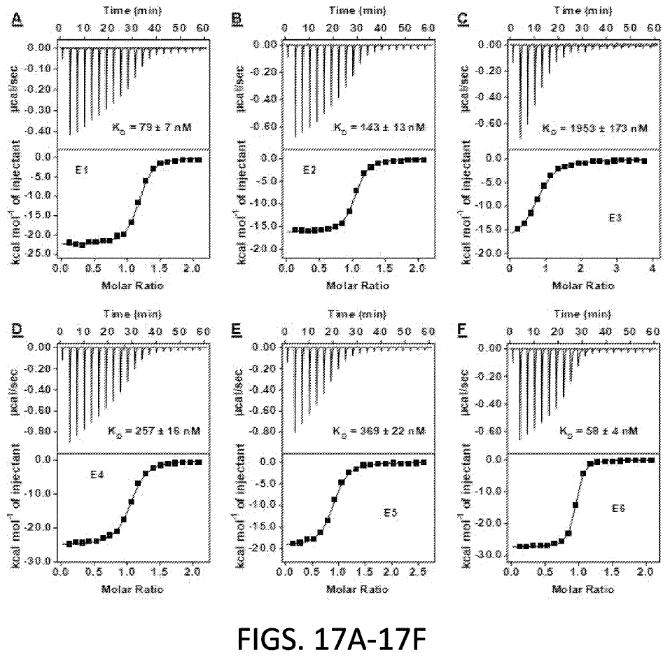

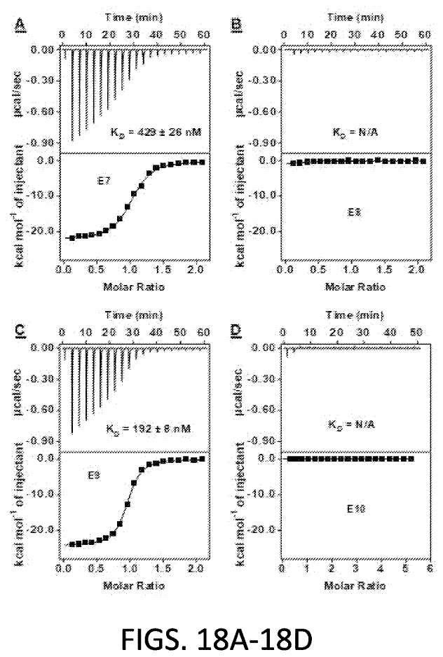

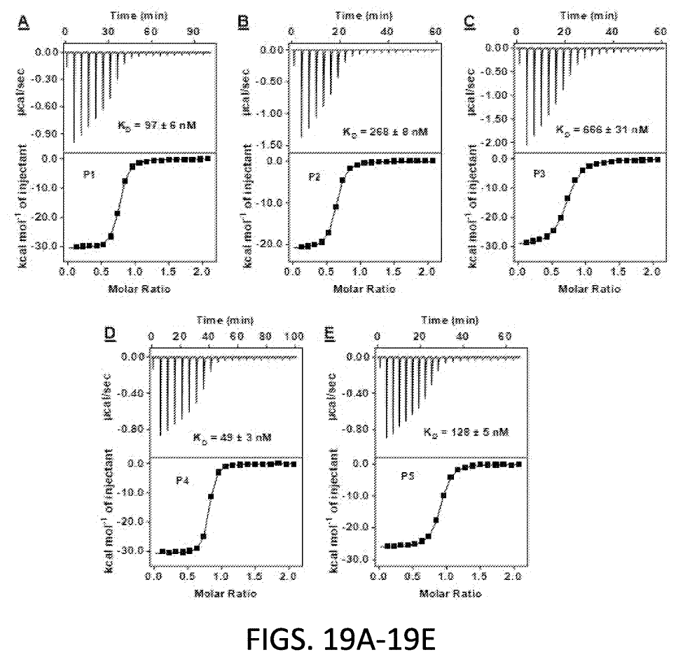

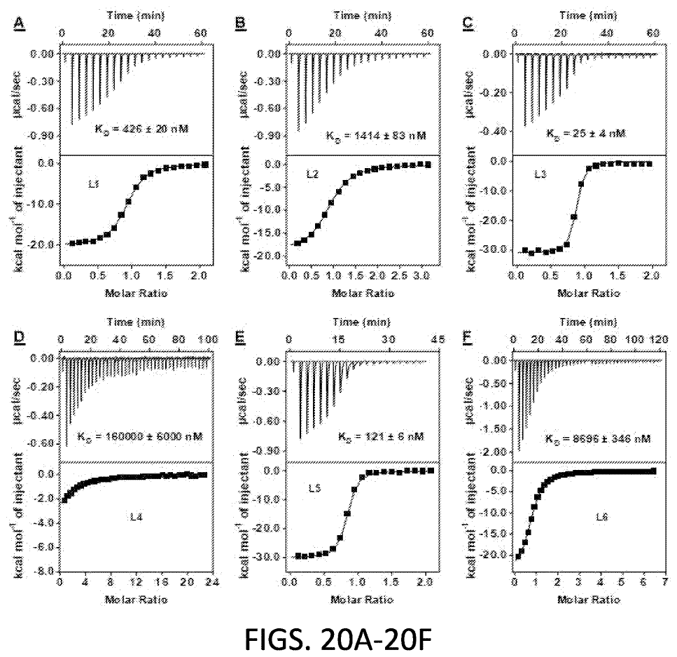

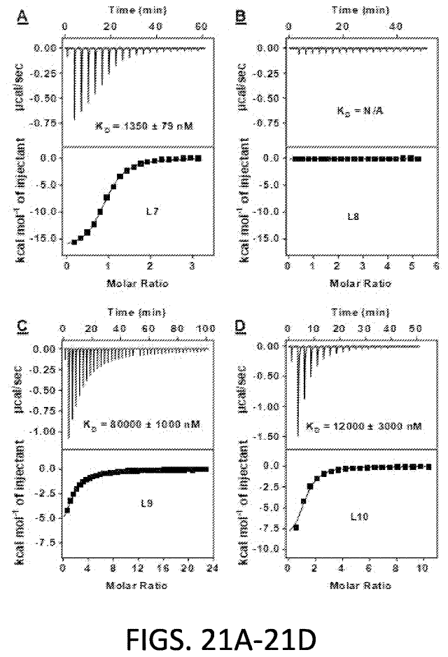

Determination of aptamer binding affinity using ITC. ITC was utilized to determine the K_{D} of these 25 aptamer candidates. The candidates exhibited affinities ranging from 25 nM to 160 μM (FIG. 2B and Table 4). Overall, 70% of exponential growth aptamer candidates (category 1, names contain ‘E’ for exponential) bound to fentanyl with high affinity in particular, E1, E2, E4, E5, E6, E7, and E9 bound tightly to fentanyl, with K_{D} of 58-429 nM; E3 had a K_{D} of 2 μM, and E8 and E10 demonstrated no affinity (FIG. 2B and FIGS. 17-18). It is possible that these latter sequences were able to survive selection due to PCR bias or non-specific dissociation. All sequences that peaked in population (category 3, aptamers named ‘P’) bound strongly to fentanyl with nanomolar affinity (K_{D} 49-666 nM) where the best binder, P4, had a K_{D} of 49 nM (FIG. 2B and FIG. 19). In contrast, most linear-growth aptamer candidates (termed ‘L’) had K_{D} ranging from 0.4-160 μM, with the exception of L3 and L5, with K_{D} of 25 and 121 nM, respectively (FIG. 2B and FIGS. 20-21). In general, all sequences could be placed into families based on their sequence similarity. Sequences in the E2 family contained a two-way-junction structure with an AT-rich hairpin loop and a variable bulge (FIG. 7A), while those in the E1 family were GT-rich (FIG. 7B). Regardless of their growth during SELEX, most high-affinity aptamers (i.e., E1, E4, E6, E9, P1, P4, P5, L3, P3, and L5) were part of the E1 family, while the only high-affinity E2 family members were E2, E5, E7, P2, and L1.

### Example 5

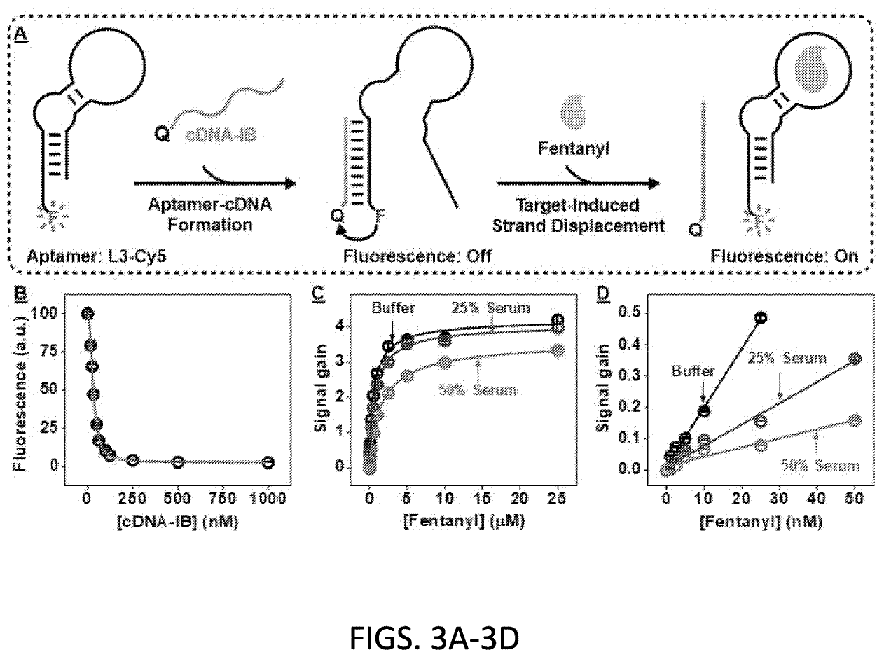

Fluorescence detection of fentanyl in serum. There is currently a critical unmet need for fentanyl detection methods that are not only accurate, sensitive, and specific, but also simple and rapid. To address this problem, an aptamer-based fluorescence strand displacement assay was developed. This detection method is simple, requiring only mixing of the aptamer and target sample, and has a rapid turnaround time. In this assay, a fluorophore-labeled aptamer is initially hybridized with a quencher-tagged cDNA, which brings the fluorophore and quencher into close proximity. In the presence of fentanyl, the aptamer binds to the target and dissociates from the cDNA, resulting in recovery of fluorescence (FIG. 3A). The highest affinity aptamer, L3 (K_{D}=25±4 nM), was chosen to develop this assay. L3 was modified with Cy5 at its 5′ end (L3-Cy5) and modified a 14-nt cDNA with an Iowa Black RQ quencher at the 3′ end (cDNA-IB). The concentration of cDNA-IB was first modified to achieve quenching efficiency of at least 80% (FIG. 3B). Afterwards, solutions of aptamer-cDNA complex were challenged with various concentrations of fentanyl in the range of 1-25,000 nM and observed target-concentration dependent increases in fluorescence (FIG. 3C). The assay had a measurable LOD of 2.5 nM in both buffer and 25% serum and 5 nM in 50% serum (FIG. 3D). Given that the average concentration of fentanyl in human blood following overdoses ranges between 30-120 nM, this assay should be sufficiently selective and sensitive to assess fentanyl overdose, especially in a high-throughput manner using a plate reader.

### Example 6

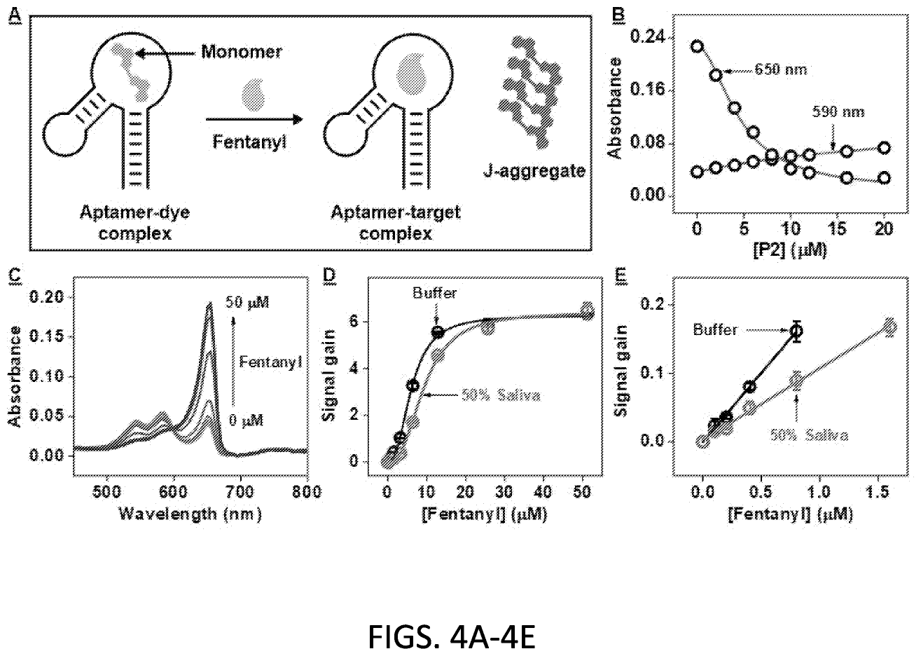

Colorimetric detection of fentanyl in saliva. Many fentanyl overdose deaths can be attributed to the consumption of drugs adulterated with fentanyl, and these could be mitigated with on-site tools for identifying exposure to this substance in a rapid, straightforward, cost-effective, and user-friendly manner from easily-attainable biosamples such as saliva. Aptamer-based dye-displacement assays are an ideal solution because they are sensitive, specific, are compatible with biosamples, and require only a single mix-and-read step. In these assays, an aptamer is complexed with a nucleic-acid-binding dye, such as cyanine dye monomers or dimers. Target binding displaces the dye into solution, which causes the dye to form aggregates due to its hydrophobicity (FIG. 4A). This results in a change in the color of the solution that can easily be observed with the naked eye, or measured with a handheld portable reader.

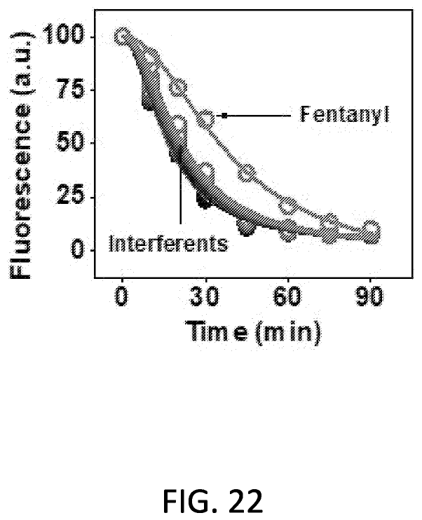

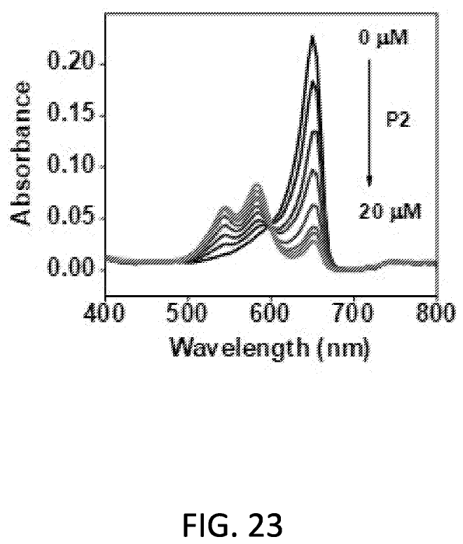

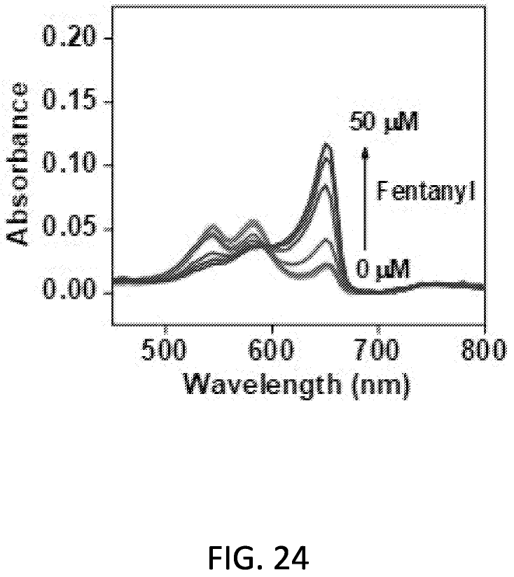

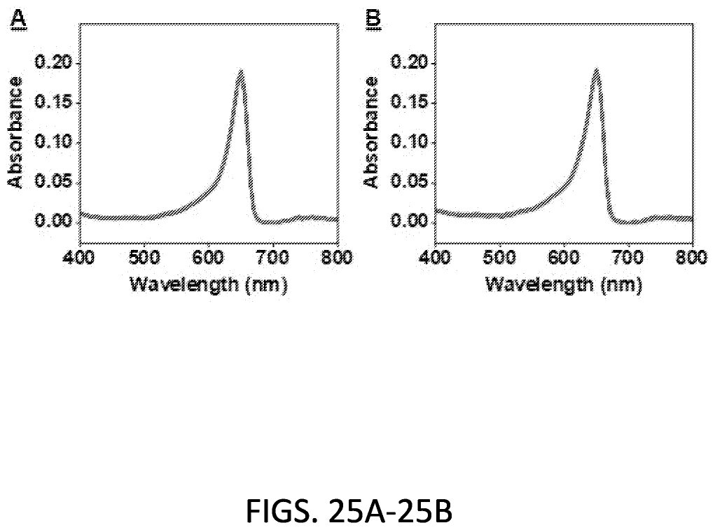

To develop such an assay for fentanyl detection, the cyanine dye MTC and the highly specific aptamer P2 were used (FIG. 22) (K_{D}=268±8 nM), which according to preliminary results, can bind and release MTC in a target-concentration dependent manner. First, to optimize the concentration of P2, various concentrations of aptamer (0-20 μM) were mixed with 1.5 μM MTC. Decreases in dye J-aggregate peak at 650 nm and increases in monomer and dimer peaks at 550 and 590 nm were observed, respectively, with increasing concentrations of aptamer (FIG. 4B and FIG. 23). Eight μM P2 was chosen as an optimal concentration for the assay given the saturation in monomer absorbance reduction observed at this aptamer concentration. The dye-aptamer complexes were challenged with various concentrations of fentanyl, which resulted in increases in J-aggregate absorbance and reduction in monomer and dimer absorbance with increasing target concentration (FIG. 4C). Using the ratiometric change of these dye forms, fentanyl was detected at as low as 100 nM, with a dynamic range of 0.1-10 μM fentanyl in buffer and 50% saliva (FIG. 24 and FIGS. 4D-4E). As expected, fentanyl concentrations as high as 50 μM did not produce any spectral changes in MTC in the absence of the P2 aptamer in either buffer or 50% saliva (FIG. 25). This assay could thus potentially be useful for ascertaining fentanyl abuse and overdose in individuals on-site, in field settings or at the roadside via a sample of oral fluid, where typical drug concentrations range from 10-350 nM.

### Example 7

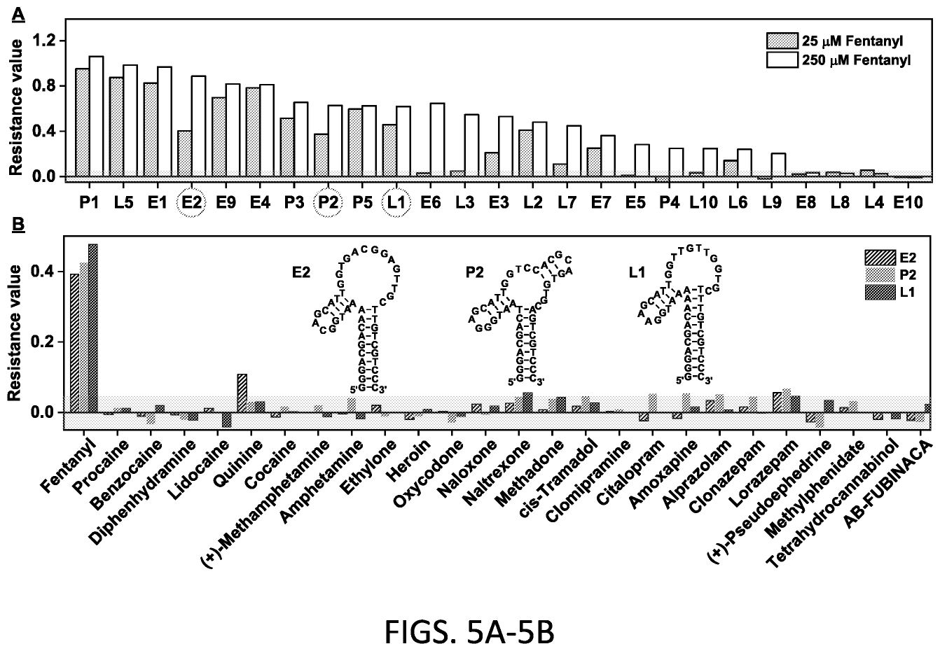

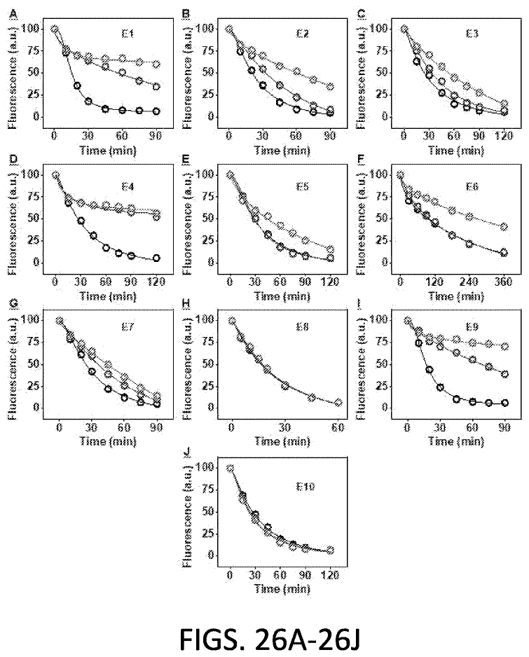

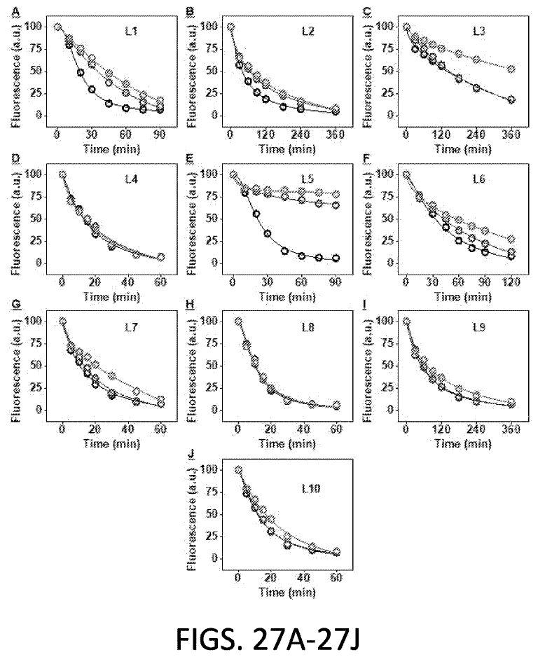

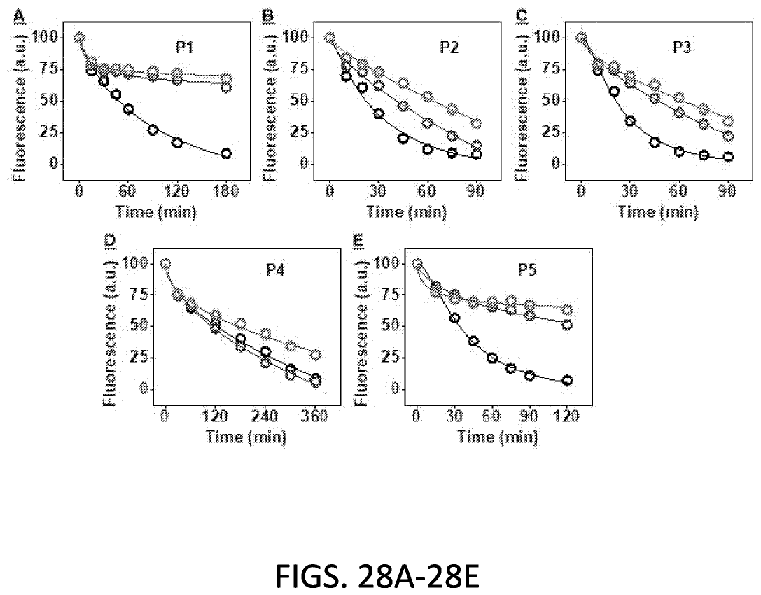

Introducing structure-switching functionality for use in an E-AB sensor. E-AB sensors are being increasingly used for small-molecule detection in biosamples due to their high selectivity, sensitivity, and rapid detection times. E-AB sensors can only operate with aptamers that undergo major conformational changes upon target binding, and in most cases such functionality must be engineered into aptamers intended for use in these sensors. Previously-reported exonuclease-based assay were used to identify an optimal structure-switching aptamer. Here, the double-stranded DNA nuclease exonuclease III (Exo III) and single-stranded nuclease exonuclease I (Exo I) digest aptamers from the 3′ terminus into mononucleotides in the absence of the target, but are unable to digest the aptamer beyond a certain point in the presence of target, typically yielding a single major truncation product. The resistance of the aptamer to enzymatic digestion is proportional to the affinity of this truncation product for its target, and the resulting truncated product typically has structure-switching functionality. The 25 aptamer candidates were digested in the presence of 0, 25, or 250 μM fentanyl and the digestion progress was monitored by taking aliquots of the reaction mixture at various times, staining with the DNA-binding dye SYBR Gold, and measuring the resulting fluorescence (FIGS. 26-28). Aptamers that bound to fentanyl tightly retained higher fluorescence in the presence of target over the whole time-course. This included aptamers P1, L5, E1, E2, E9, E4, P3, P2, P5, and L1, which all displayed nanomolar affinity based on ITC. The digestion kinetics were then analyzed using the AUC parameter, and used this to compute the resistance value (Rᵥₐₗᵤₑ) as a metric for evaluating aptamer-ligand binding strength (FIG. 5A), where Rᵥₐₗᵤₑ=(AUC_{target}/AUC_{blank})−1. A greater Rᵥₐₗᵤₑ indicates stronger aptamer-ligand binding, whereas a Rᵥₐₗᵤₑ of 0 indicates that the aptamer and ligand have no affinity for each other. The other aptamers showed little to no enzymatic inhibition in the presence of fentanyl, which demonstrated that their truncation products had poor target-binding affinity. These sequences were therefore omitted from further testing.

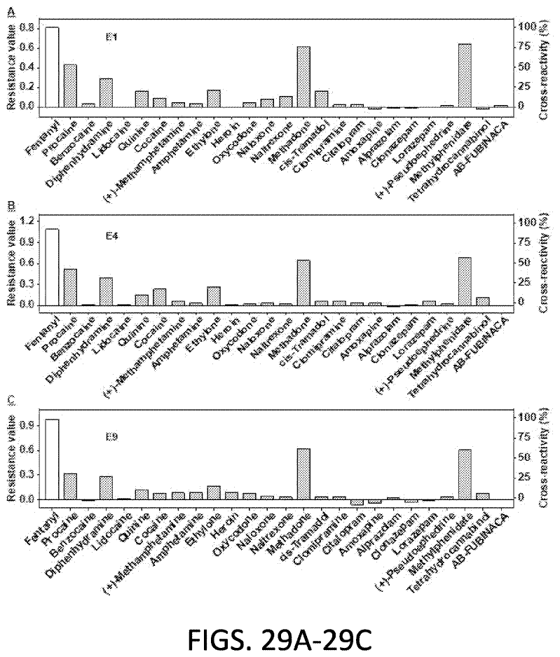

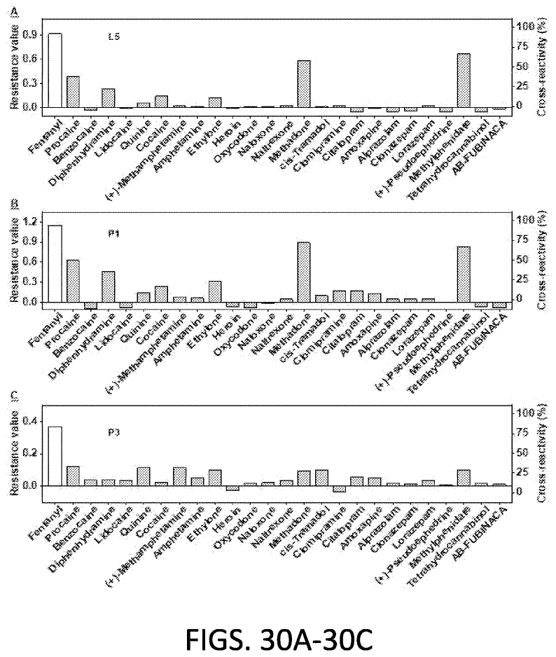

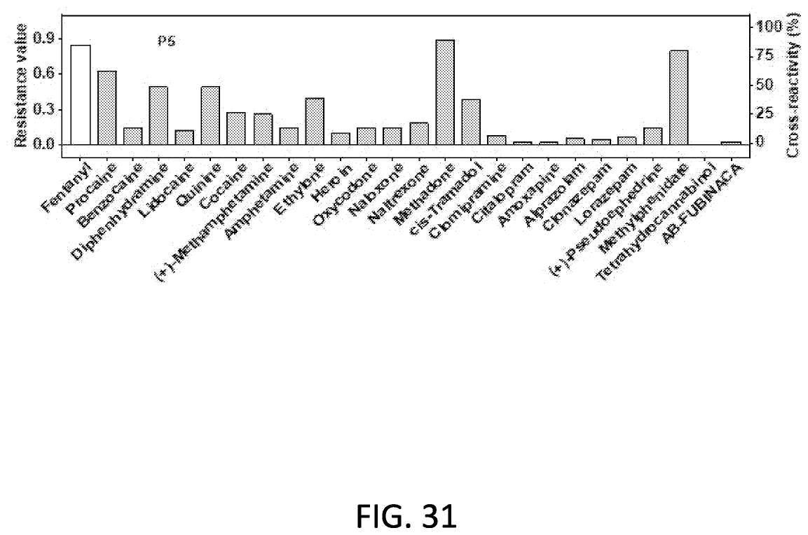

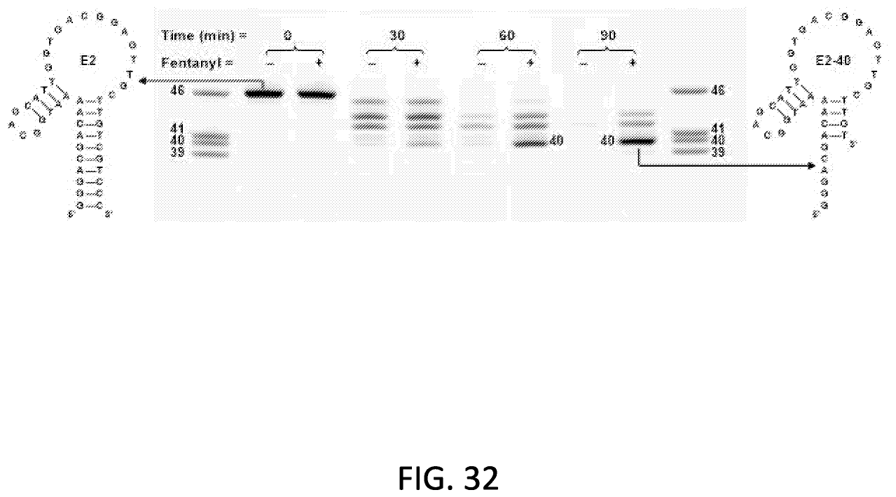

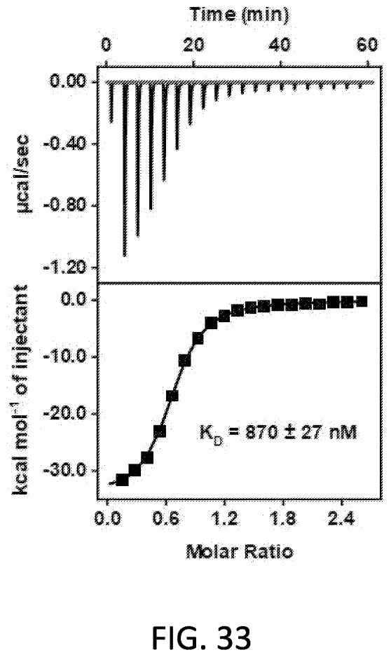

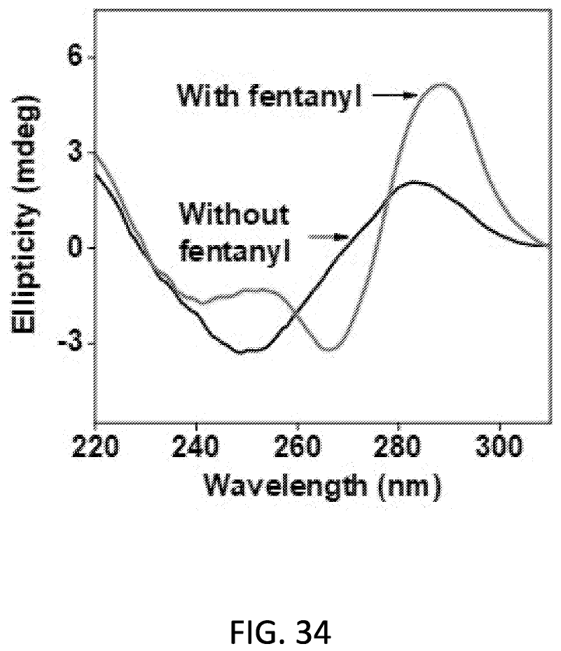

Next, the specificity of the top ten aptamer sequences against the counter-targets employed in counter-SELEX were determined using the exonuclease fluorescence assay. In general, it was found that aptamers from the E1 family (e.g., P1, L5, E1, E9, E4, P3, and P5) displayed cross-reactivity to cocaine, procaine, diphenhydramine, ethylone, methylphenidate, and methadone (FIGS. 29-31). In contrast, E2 family members had superior specificity; L1, E2, and P2 had <10% cross-reactivity to any tested compound, except for E2 which had ~25% cross-reactivity for quinine (FIG. 5B). Given that E2 had the best combination of affinity and specificity, this aptamer was chosen for further sensor development. First, the identity of its exonuclease truncation product was determined by performing polyacrylamide gel electrophoresis analysis of the digested aptamer. It was observed that the aptamer was truncated 6 nt from its 3′ end to form a 40-nt product (FIG. 32). The remaining 5′ overhang of this aptamer was removed and the 34-nucleotide construct, termed E2-34, was synthesized. Using ITC, it was determined that E2-34 bound to fentanyl with a K_{D} of 870±27 nM, a 14-fold higher value than the K_{D} of the parent aptamer (FIG. 33). Circular dichroism spectroscopy was then performed to determine whether the aptamer underwent conformational changes upon target binding (FIG. 34). The aptamer by itself yielded a spectrum with a broad positive peak spanning from 260 nm to 290 nm and a negative peak at 250 nm, indicating the presence of B-form DNA. Upon the addition of fentanyl, the positive peak grew in intensity and its peak shifted to ~290 nm, the negative peak decreased in intensity, and a new negative peak formed at ~265 nm. This spectrum is indicative of the formation of an anti-parallel G-quadruplex, and provides clear confirmation of a fentanyl-binding induced conformational change.

### Example 8

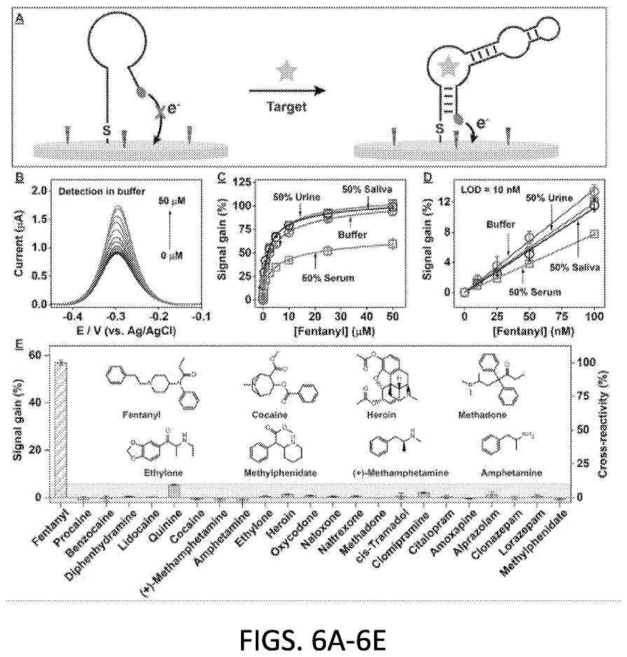

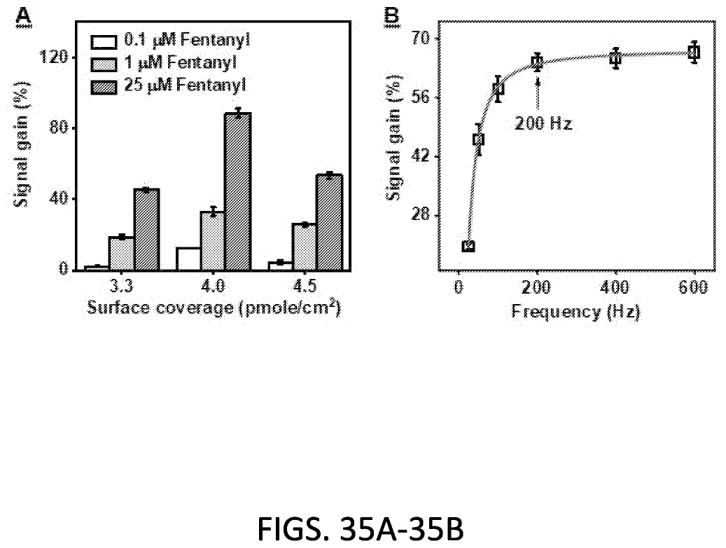

Development of E-AB sensor for fentanyl detection in biological samples. E2-34 was synthesized with a 3′-methylene blue tag and a 5′-thiol group and utilized this construct (E2-34-MB) to fabricate E-AB sensors. First, E2-34-MB was immobilized onto a gold working electrode using previously-reported target-assisted immobilization strategy. In this strategy, the aptamer is first complexed with the target and then immobilized onto the electrode to provide sufficient spacing for aptamer target binding and folding. E-AB sensors made using this strategy have improved signal-to-noise ratios and LOD relative to those made using conventional aptamer immobilization approaches. E-AB sensors are folding-based sensors, such that in the absence of target, the electrode-bound aptamers are unfolded and have their methylene blue redox tag positioned far from the electrode surface, resulting in a small background current. When challenged with the target, aptamer-target binding triggers a conformational change that brings the methylene blue tag closer to the electrode surface, thus increasing the redox current in a target concentration-dependent manner (FIG. 6A). The surface coverage of the sensor was first optimized by immobilizing different amounts of aptamer on the electrode and found that electrodes with aptamer density of 4.0 pmol/cm² yielded the highest signal gain (FIG. 35A). The square-wave measurement frequency was also optimized to a final value of 200 Hz (FIG. 35B). After optimizing the E-AB sensor, it was challenged with 0-50 μM fentanyl and increased current was observed with increasing target concentrations (FIG. 6B), obtaining a linear range from 0-100 nM and a LOD of 10 nM in buffer (FIGS. 6C-D).

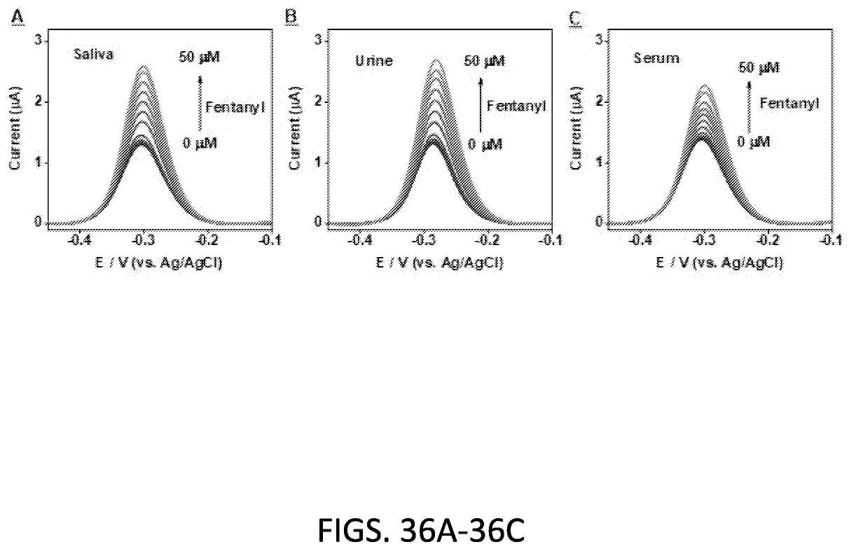

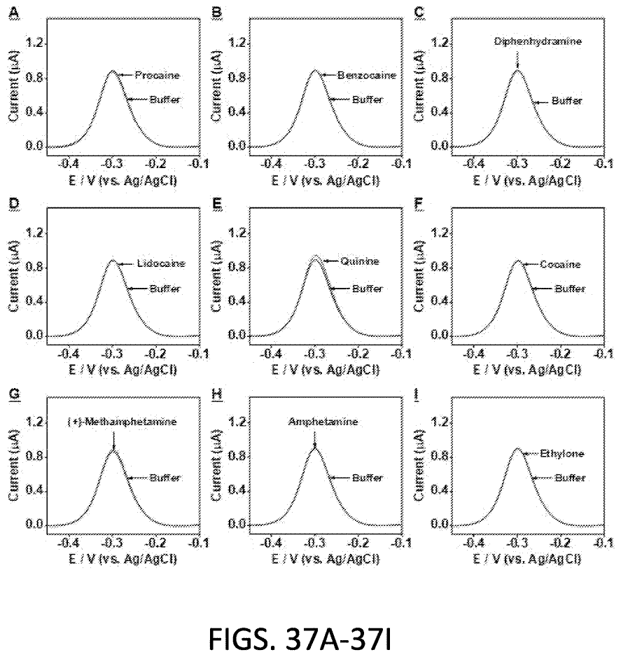

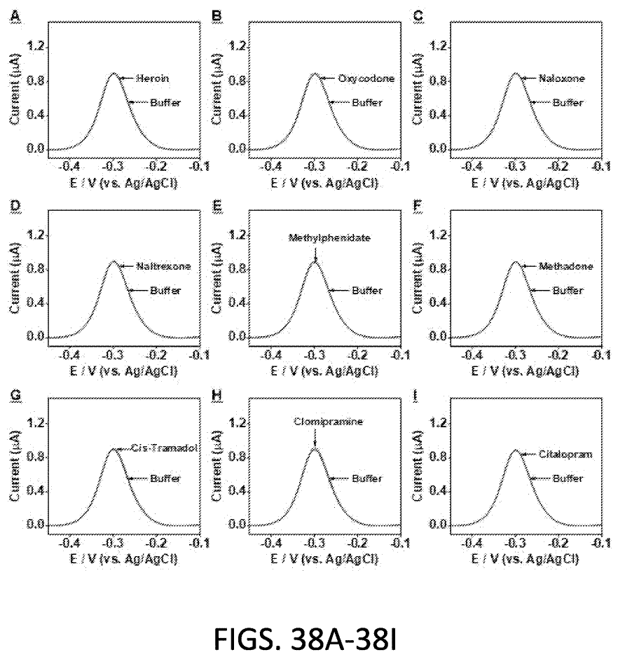

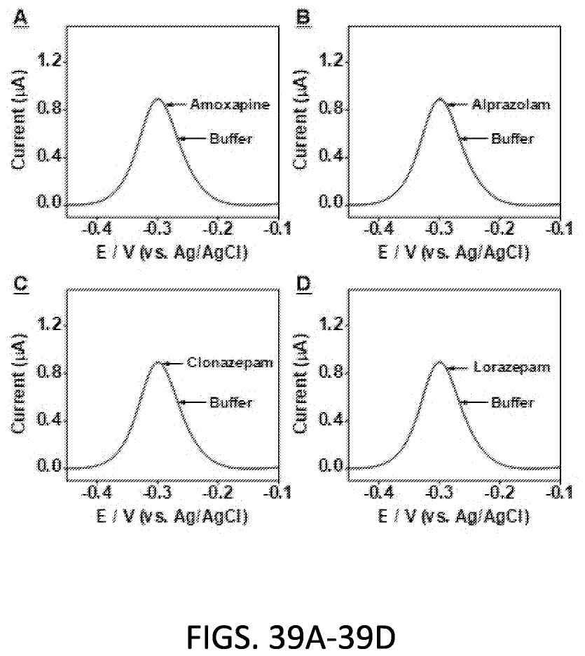

Impressively, the sensor demonstrated similar sensing performance in buffer, 50% human saliva, and 50% human urine (FIGS. 36A-36B). Some signal suppression was observed in 50% human serum, but the sensor still retained excellent performance with a LOD of 10 nM fentanyl and a robust signal-to-noise ratio (FIGS. 6C-D) (FIG. 36C). Finally, the cross-reactivity of the E-AB sensor to 22 different interferents was assessed at 5- to 10-fold higher concentrations relative to fentanyl, and <10% cross-reactivity to quinine and minimal cross-reactivity to all other substances was observed (FIG. 6D) (FIGS. 37-39). These results demonstrate that the E-AB sensor is sufficiently sensitive to detect fentanyl at low concentration in biological matrices without any significant response to interferents. Given that in the context of overdose cases, the concentration of fentanyl is typically 10-350 nM in human blood, saliva, and urine, the sensor is suitable for rapid and accurate assessment of fentanyl overdose on-site in medical settings such as ambulances and emergency departments.

What is claimed is:

1. A single-stranded nucleic acid molecule capable of binding fentanyl, or a derivative or analog thereof, comprising a nucleic acid sequence that is at least 50% identical to the following: X₁GCTTGGX₂TAGGTCGGGX₃TTGGGTX₄CGGX₅X₆; wherein X₁ is A, T, C, or G; X₂ is A, T, C, or G; X₃ is A, T, C, or G; X₄ is A, T, C, or G; X₅ is A, T, C, or G; and X₆ is A, T, C, or G (SEQ ID NO: 1).

2. The nucleic acid molecule of claim 1, wherein X₁ is T or A; X₂ is G or T; X₃ is T or C, X₄ is T or G, X₅ is C or A; and X₆ is G or T (SEQ ID NO: 2).

3. The nucleic acid molecule of claim 1, wherein the nucleic acid molecule comprises a nucleic acid sequence that is at least 50% identical to any of SEQ ID NOs: 5, 8, 10, 13, 17, 19, 25, 27, 28, and 29.

4. The nucleic acid molecule of claim 1, wherein the nucleic acid molecule comprises a K_{D} that is less than about 500 nM.

5. A single-stranded nucleic acid molecule capable of binding fentanyl, or a derivative or analog thereof, comprising a nucleic acid sequence that is at least 50% identical to the following: X₁X₂AATGGX₃AGCATTGGTX₄X₅X₆X₇X₈X₉X₁₀X₁X₁₂X₁₃X₁₄X₁₅X₁₆; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ A, T, C, or G; X₁₁ is A, T, C, or G; X₁₂ is A, T, C, or G; X₁₃ is A, T, C, or G; X₁₄ is A, T, C, or G; X₁₅ is A, T, C, or G; and X₁₆ is A, T, C, or G (SEQ ID NO: 3).

6. The nucleic acid molecule of claim 5, wherein X₁ is A or G; X₂ is A or G; X₃ is C or A, X₄ is G or T, X₅ is A or G; X₆ is C or G; X₇ is T or G; X₈ is G or T; X₉ is G or A; X₁₀ G or T; X₁₁ is T or G; X₁₂ is T or C; X₁₃ is G or T; X₁₄ is C or T; X₁₅ is T or C; and X₁₆ is T or C (SEQ ID NO: 4).

7. The nucleic acid molecule of claim 5, wherein the nucleic acid molecule comprises a nucleic acid sequence that is at least 50% identical to any of SEQ ID NOs: 6, 9, 11, 15, and 26.

8. The nucleic acid molecule of claim 5, wherein the nucleic acid molecule comprises a K_{D} that is less than about 500 nM.

9. The nucleic acid molecule of claim 1 or claim 5, wherein the nucleic acid molecule is capable of binding fentanyl, or a derivative or analog thereof, under physiological conditions.

10. The nucleic acid molecule of claim 1 or claim 5, wherein the nucleic acid molecule comprises a detection moiety.

11. The nucleic acid molecule of claim 1 or claim 5, wherein the nucleic acid molecule is in solution or attached to a substrate.

12. The nucleic acid molecule of claim 1 or claim 5, wherein the fentanyl derivative or analog is selected from the group consisting of: acetyl fentanyl, furanyl fentanyl, acrylfentanyl, butyryl fentanyl, valeryl fentanyl, cyclopropyl fentanyl, methoxyacetyl fentanyl, p-fluoro fentanyl, o-methyl furanyl fentanyl, o-methoxy furanyl fentanyl, p-methoxy furanyl fentanyl, p-methoxy butyryl fentanyl, FIBF, alpha-methyl thiofentanyl, 3,4-dimethoxy fentanyl, beta-hydroxy thiofentanyl, cis-3-methyl fentanyl, trans-3-methyl fentanyl, cis-3-methyl thiofentanyl, trans-3-methyl thiofentanyl, sufentanil, alfentanil, remifentanil, benzylfentanyl, N-benzyl furanyl norfentanyl, N-benzyl m-fluoro norfentanyl, N-benzyl o-fluoro norfentanyl, N-benzyl phenyl norfentanyl, norfentanyl, and 4-ANPP.

13. A vector comprising the nucleic acid molecule of claim 1 or claim 5.

14. A method of detecting fentanyl, or a derivative or analog thereof, the method comprising:
combining the nucleic acid molecule of claim 1 or claim 5 comprising a fluorescent moiety with a quencher-labeled nucleic acid molecule that is at least partially complementary to the nucleic acid molecule of claim 1 or claim 5 to form a quenched composition; and
exposing the quenched composition to a sample comprising or suspected of comprising fentanyl, or a derivative or analog thereof;
wherein presence of the fentanyl, or a derivative or analog thereof, in the sample displaces the quencher-labeled nucleic acid molecule, thereby producing a fluorescent signal proportional to the concentration of the fentanyl, or a derivative or analog thereof, in the sample.

15. A method of detecting fentanyl, or a derivative or analog thereof, the method comprising:
combining the nucleic acid molecule of claim 1 or claim 5 with a reporter compound that binds to the nucleic acid molecule of claim 1 or claim 5 to form a complexed composition; and
exposing the complexed composition to a sample comprising or suspected of comprising fentanyl, or a derivative or analog thereof;
wherein presence of the fentanyl, or a derivative or analog thereof, in the sample displaces the reporter compound, thereby allowing the reporter compound to form detectable aggregates proportional to the concentration of the fentanyl, or a derivative or analog thereof, in the sample.

16. A method of detecting fentanyl, or a derivative or analog thereof, the method comprising:
immobilizing the nucleic acid molecule of claim 1 or claim 5 to an electrically conductive substrate, wherein the nucleic acid molecule of claim 1 or claim 5 comprise a redox tag, to form a detection sensor; and
exposing the detection sensor to a sample comprising or suspected of comprising fentanyl, or a derivative or analog thereof;
wherein presence of the fentanyl, or a derivative or analog thereof, in the sample binds the nucleic acid molecule of claim 1 or claim 5, thereby producing an electrochemical signal proportional to the concentration of the fentanyl, or a derivative or analog thereof, in the sample.

17. The method of claim 14, wherein the sample is a biological sample from a human subject.

18. The method of claim 17, wherein the biological sample is a saliva sample, a urine sample, a blood sample, a serum sample, a plasma sample, a fecal sample, a CSF sample, or a tissue sample.

19. The method of claim 14, wherein the fentanyl derivative or analog is selected from the group consisting of: acetyl fentanyl, furanyl fentanyl, acrylfentanyl, butyryl fentanyl, valeryl fentanyl, cyclopropyl fentanyl, methoxyacetyl fentanyl, p-fluoro fentanyl, o-methyl furanyl fentanyl, o-methoxy furanyl fentanyl, p-methoxy furanyl fentanyl, p-methoxy butyryl fentanyl, FIBF, alpha-methyl thiofentanyl, 3,4-dimethoxy fentanyl, beta-hydroxy thiofentanyl, cis-3-methyl fentanyl, trans-3-methyl fentanyl, cis-3-methyl thiofentanyl, trans-3-methyl thiofentanyl, sufentanil, alfentanil, remifentanil, benzylfentanyl, N-benzyl furanyl norfentanyl, N-benzyl m-fluoro norfentanyl, N-benzyl o-fluoro norfentanyl, N-benzyl phenyl norfentanyl, norfentanyl, and 4-ANPP.

20. A method of identifying a nucleic acid aptamer capable of binding fentanyl, or a derivative or analog thereof, the method comprising:
(i) exposing a library of candidate aptamers to fentanyl, or a derivative or analog thereof;
(ii) collecting candidate aptamers exhibiting rapid target association kinetics; and
(iii) repeating steps (i) and (ii) for at least one addition round of selection to generate a plurality of high-affinity candidate aptamers.

21-23. (canceled)


SEQUENCE LISTING (39 sequences)

SEQ ID NO: 1 - DNA, 30 bp, Synthetic construct
  source 1..30: mol_type = other DNA
ngcttggntaggtcgggnttgggtncggnn

SEQ ID NO: 2 - DNA, 30 bp, Synthetic construct
  source 1..30: mol_type = other DNA
wgcttggktaggtcgggyttgggtkcggmk

SEQ ID NO: 3 - DNA, 30 bp, Synthetic construct
  source 1..30: mol_type = other DNA
nnaatggnagcattggtnnnnnnnnnnnnn

SEQ ID NO: 4 - DNA, 30 bp, Synthetic construct
  source 1..30: mol_type = other DNA
rraatggmagcattggtkrskkrkkykyyy

SEQ ID NO: 5 - DNA, 46 bp, Synthetic construct
  source 1..46: mol_type = other DNA
gggacgactgcttgggtaggtcgggcttgggttcggcggtcgtccc

SEQ ID NO: 6 - DNA, 46 bp, Synthetic construct
  source 1..46: mol_type = other DNA
gggacgacaaaatggcagcattggtgacggagttgcttgtcgtccc

SEQ ID NO: 7 - DNA, 46 bp, Synthetic construct
  source 1..46: mol_type = other DNA
gggacgacaggtcggtcagtggtagggtaggtctatctgtcgtccc

SEQ ID NO: 8 - DNA, 46 bp, Synthetic construct
  source 1..46: mol_type = other DNA
gggacgacagcttggtttggtcggggtagggtccggatgtcgtccc

SEQ ID NO: 9 - DNA, 46 bp, Synthetic construct
  source 1..46: mol_type = other DNA
gggacgacgtaatgggagcattggtcacgcaggtgcacgtcgtccc

SEQ ID NO: 10 - DNA, 46 bp, Synthetic construct
  source 1..46: mol_type = other DNA
gggacgacggttggcattgggtcgggtgtgggtgcggcgtcgtccc

SEQ ID NO: 11 - DNA, 46 bp, Synthetic construct
  source 1..46: mol_type = other DNA
gggacgacagaatggcagcattggtgagttatttgcctgtcgtccc

SEQ ID NO: 12 - DNA, 47 bp, Synthetic construct
  source 1..47: mol_type = other DNA
gggacgacaaaaatggaagcattggtgacggagttgcttgtcgtccc

SEQ ID NO: 13 - DNA, 46 bp, Synthetic construct
  source 1..46: mol_type = other DNA
gggacgacagcttggtctggtcggggtagggttcggatgtcgtccc

SEQ ID NO: 14 - DNA, 48 bp, Synthetic construct
  source 1..48: mol_type = other DNA
gggacgacaaaaatggaagcattggtccacgcagtgtgcagtcgtccc

SEQ ID NO: 15 - DNA, 46 bp, Synthetic construct
  source 1..46: mol_type = other DNA
gggacgacaaaaatggaagcattggttgttggtgctttgtcgtccc

SEQ ID NO: 16 - DNA, 46 bp, Synthetic construct
  source 1..46: mol_type = other DNA
gggacgacgtgtaggtgtgcacccgcagggctctgcaggtcgtccc

SEQ ID NO: 17 - DNA, 45 bp, Synthetic construct
  source 1..45: mol_type = other DNA
gggacgacggttggcttgggtcgggtgtgggtgcggcgtcgtccc

SEQ ID NO: 18 - DNA, 46 bp, Synthetic construct
  source 1..46: mol_type = other DNA
gggacgacaaaaatggaagcattggttgttggtgccttgtcgtccc

SEQ ID NO: 19 - DNA, 46 bp, Synthetic construct
  source 1..46: mol_type = other DNA
gggacgacagcttggcttggtcggggtagggttcggatgtcgtccc

SEQ ID NO: 20 - DNA, 46 bp, Synthetic construct
  source 1..46: mol_type = other DNA
gggacgacagcgtggtttggtcggggtagggttcggatgtcgtccc

SEQ ID NO: 21 - DNA, 46 bp, Synthetic construct
  source 1..46: mol_type = other DNA
gggacgacaaaaatggaagcattggttgtcggtgctttgtcgtccc

SEQ ID NO: 22 - DNA, 46 bp, Synthetic construct
  source 1..46: mol_type = other DNA
gggacgacaaaaatggaagcattggttgttggcgctttgtcgtccc

SEQ ID NO: 23 - DNA, 46 bp, Synthetic construct
  source 1..46: mol_type = other DNA
gggacgacagcttggtttggttggggtagggttcggatgtcgtccc

SEQ ID NO: 24 - DNA, 47 bp, Synthetic construct
  source 1..47: mol_type = other DNA
gggacgacaaaaaatggaagcattggttgttggtgctttgtcgtccc

SEQ ID NO: 25 - DNA, 46 bp, Synthetic construct
  source 1..46: mol_type = other DNA
gggacgacagcttggtttggtcggggtagggttcggatgtcgtccc

SEQ ID NO: 26 - DNA, 46 bp, Synthetic construct
  source 1..46: mol_type = other DNA
gggacgactaatgggagcattggtccacgcagtgtgcagtcgtccc

SEQ ID NO: 27 - DNA, 46 bp, Synthetic construct
  source 1..46: mol_type = other DNA
gggacgacaatgggtggtgttagcgagtgtctttccttgtcgtccc

SEQ ID NO: 28 - DNA, 46 bp, Synthetic construct
  source 1..46: mol_type = other DNA
gggacgacggttggcttggggtcgggtgtgggtgcggcgtcgtccc

SEQ ID NO: 29 - DNA, 46 bp, Synthetic construct
  source 1..46: mol_type = other DNA
gggacgactgcttgggtaggtcgggtttgggttcggcggtcgtccc

SEQ ID NO: 30 - DNA, 73 bp, Synthetic construct
  source 1..73: mol_type = other DNA
ggaggctctcgggacgacnnnnnnnnnnnnnnnnnnnnnnnnnnnnnngtcgtcccgcctttaggatttacag

SEQ ID NO: 31 - DNA, 18 bp, Synthetic construct
  source 1..18: mol_type = other DNA
gtcgtcccgagagccata

SEQ ID NO: 32 - DNA, 18 bp, Synthetic construct
  source 1..18: mol_type = other DNA
ggaggctctcgggacgac

SEQ ID NO: 33 - DNA, 25 bp, Synthetic construct
  source 1..25: mol_type = other DNA
ctgtaaatcctaaaggcgggacgac

SEQ ID NO: 34 - DNA, 96 bp, Synthetic construct
  source 1..96: mol_type = other DNA
acactctttccctacacgacgctcttccgatctnnnnnnnnnnnnnnnnnnnnnnnnnnnnnnnnnnnnnnnnnnnnnggaggctctcgggacgac

SEQ ID NO: 35 - DNA, 100 bp, Synthetic construct
  source 1..100: mol_type = other DNA
gactggagttcagacgtgtgctcttccgatctnnnnnnnnnnnnnnnnnnnnnnnnnnnnnnnnnnnnnnnnnnnctgtaaatcctaaaggcgggacgac

SEQ ID NO: 36 - DNA, 34 bp, Synthetic construct
  source 1..34: mol_type = other DNA
acaaaatggcagcattggtgacggagttgcttgt

SEQ ID NO: 37 - DNA, 53 bp, Synthetic construct
  source 1..53: mol_type = other DNA
tggctctcgggacgacggttggcttgggtcgggtgtgggtgcggcgtcgtccc

SEQ ID NO: 38 - DNA, 14 bp, Synthetic construct
  source 1..14: mol_type = other DNA
gtcgtcccgagagc

SEQ ID NO: 39 - DNA, 34 bp, Synthetic construct; identical to SEQ ID NO: 36
  source 1..34: mol_type = other DNA